SEQUENCE LISTING (13 sequences)

SEQ ID NO: 1 - DNA, 21 bp, Homo sapiens
gactatatcgtgccctgcatg

SEQ ID NO: 2 - DNA, 21 bp, Homo sapiens
gccactctttgaccggttgct

SEQ ID NO: 3 - DNA, 21 bp, Homo sapiens
aacagtcgcgtttgcgactgg

SEQ ID NO: 4 - DNA, 42 bp, Homo sapiens
aaacagatcatccgcaaacacgtgtgaggcggtagtaggaca

SEQ ID NO: 5 - DNA, 21 bp, Homo sapiens
aacatcgagccactctttgac

SEQ ID NO: 6 - DNA, 21 bp, Homo sapiens
tccttggcatcaaaataccag

SEQ ID NO: 7 - DNA, 23 bp, Homo sapiens
aagacgatcagataccgtcgtag

SEQ ID NO: 8 - DNA, 23 bp, Homo sapiens
gtttcagctttgcaaccatactc

SEQ ID NO: 9 - DNA, 1248 bp, Homo sapiens
atggactacaaagacgatgacgacatggacagcccgtgccagccgcagcccctaagtcaggctctccctcagttaccagggtcttcgtcagagcccttggagcctgagcctggccgggccaggatgggagtggagagttacctgccctgtcccctgctcccctcctaccactgtccaggagtgcctagtgaggcctcggcagggagtgggacccccagagccacagccacctctaccactgccagccctcttcgggacggttttggcgggcaggatggtggtgagctgcggccgctgcagagtgaaggcgctgcagcgctggtcaccaaggggtgccagcgattggcagcccagggcgcacggcctgaggcccccaaacggaaatgggccgaggatggtggggatgccccttcacccagcaaacggccctgggccaggcaagagaaccaggaggcagagcgggagggtggcatgagctgcagctgcagcagtggcagtggtgaggccagtgctgggctgatggaggaggcgctgccctctgcgcccgagcgcctggccctggactatatcgtgccctgcatgcggtactacggcatctgcgtcaaggacagcttcctgggggcagcactgggcggtcgcgtgctggccgaggtggaggccctcaaacggggtgggcgcctgcgagacgggcagctagtgagccagagggcgatcccaccgcgcagcatccgtggggaccagattgcctgggtggaaggccatgaaccaggctgtcgaagcattggtgccctcatggcccatgtggacgccgtcatccgccactgcgcagggcggctgggcagctatgtcatcaacgggcgcaccaaggccatggtggcgtgttacccaggcaacgggctcgggtacgtaaggcacgttgacaatccccacggcgatgggcgctgcatcacctgtatctattacctgaatcagaactgggacgttaaggtgcatggcggcctgctgcagatcttccctgagggccggcccgtggtagccaacatcgagccactctttgaccggttgctcattttctggtctgaccggcggaacccccacgaggtgaagccagcctatgccaccaggtacgccatcactgtctggtattttgatgccaaggagcgggcagcagccaaagacaagtatcagctagcatcaggacagaaaggtgtccaagtacctgtatcacagccgcctacgcccacctag

SEQ ID NO: 10 - DNA, 21 bp, Homo sapiens
ggctacgtccaggagcgcacc

SEQ ID NO: 11 - DNA, 2110 bp, Homo sapiens
gctttcccctgcctgcctgtctctagtttctctcacatcccttttttttttcctttctctagccaccctgaagggtcccttcccaagcccttagggaccgcagaggacttggggaccagcaagcaacccccagggcacgagaagagctcttgctgtctgccctgcctcaccctgccccacaccaggcccggtggcccccagctgcatcaagtggaggcggaggaggaggcggaggagggtggcaccatgggcccgggcggtgccctccatgcccgggggatgaagacactgctgccatggacagcccgtgccagccgcagcccctaagtcaggctctccctcagttaccagggtcttcgtcagagcccttggagcctgagcctggccgggccaggatgggagtggagagttacctgccctgtcccctgctcccctcctaccactgtccaggagtgcctagtgaggcctcggcagggagtgggacccccagagccacagccacctctaccactgccagccctcttcgggacggttttggcgggcaggatggtggtgagctgcggccgctgcagagtgaaggcgctgcagcgctggtcaccaaggggtgccagcgattggcagcccagggcgcacggcctgaggcccccaaacggaaatgggccgaggatggtggggatgccccttcacccagcaaacggccctgggccaggcaagagaaccaggaggcagagcgggagggtggcatgagctgcagctgcagcagtggcagtggtgaggccagtgctgggctgatggaggaggcgctgccctctgcgcccgagcgcctggccctggactatatcgtgccctgcatgcggtactacggcatctgcgtcaaggacagcttcctgggggcagcactgggcggtcgcgtgctggccgaggtggaggccctcaaacggggtgggcgcctgcgagacgggcagctagtgagccagagggcgatcccgccgcgcagcatccgtggggaccagattgcctgggtggaaggccatgaaccaggctgtcgaagcattggtgccctcatggcccatgtggacgccgtcatccgccactgcgcagggcggctgggcagctatgtcatcaacgggcgcaccaaggccatggtggcgtgttacccaggcaacgggctcgggtacgtaaggcacgttgacaatccccacggcgatgggcgctgcatcacctgtatctattacctgaatcagaactgggacgttaaggtgcatggcggcctgctgcagatcttccctgagggccggcccgtggtagccaacatcgagccactctttgaccggttgctcattttctggtctgaccggcggaacccccacgaggtgaagccagcctatgccaccaggtacgccatcactgtctggtattttgatgccaaggagcgggcagcagccaaagacaagtatcagctagcatcaggacagaaaggtgtccaagtacctgtatcacagccgcctacgcccacctagtggccagtcccagagccgcatggcagacagcttaaatgacttcaggagagccctgggcctgtgctggctgctccttccctgccaccgctgctgcttctgactttgcctctgtcctgcctggtgtggagggctctgtctgttgctgaggaccaaggaggagaagagacctttgctgccccatcatgggggctggggttgtcacctggacagggggcagccgtggaggccaccgttaccaactgaagctgggggcctgggtcctaccctgtctggtcatgaccccattaggtatggagagctgggaggaggcattgtcacttcccaccaggatgcaggacttggggttgaggtgagtcatggcctcttgctggcaatggggtgggaggagtacccccaagtcctctcactcctccagcctggaatgtgaagtgactccccaacccctttggccatggcaggcaccttttggactgggctgccactgcttgggcagagtaaaaggtgccaggaggagcatgggtgtggaagtcctgtcagccaagaaataaaagtttacctcagagctgcaaaaaaaaaaaaaaaaaaaaaaa

SEQ ID NO: 12 - DNA, 19 bp, Homo sapiens
agctccttctactgctgca

SEQ ID NO: 13 - DNA, 19 bp, Homo sapiens
caggttatgttcgccacgt

(19) United States
(12) Patent Application Publication
(10) Pub. No.: US 2011/0275697 A1
(43) Pub. Date: Nov. 10, 2011

(54) REGULATION OF CYCLIN D

(75) Inventors: William G. Kaelin, JR., Boston, MA (US); Archana Bommi-Reddy, Boston, MA (US); Jinming Gu, Brookline, MA (US)

(73) Assignee: DANA FARBER CANCER INSTITUTE, INC., BOSTON, MA (US)

(21) Appl. No.: 12/735,738

(22) PCT Filed: Feb. 12, 2009
(86) PCT No.: PCT/US2009/000920

§ 371 Date: Apr. 19, 2011

Related U.S. Application Data
(60) Provisional application No. 61/065,440, filed on Feb. 12, 2008.

(51) Int. Cl. A61K 31/713; G01N 33/53; A61P 35/00; C12Q 1/26

(52) U.S. Cl. 514/44 A; 435/25; 435/7.1

(57) ABSTRACT

The present invention provides methods for modulating the level or activity of cyclin D by inhibiting EGLN2 expression or activity. The methods are particularly useful for treating or preventing a disorder associated with elevated cyclin D levels or activity, such as cancer.

### FIELD OF THE INVENTION

The present invention provides methods for modulating the level or activity of cyclin D by inhibiting EGLN2 expression or activity. The methods are particularly useful for treating or preventing a disorder associated with elevated cyclin D levels or activity, such as cancer.

### INCORPORATION OF SEQUENCE LISTING

The computer readable sequence listing and sequence descriptions therein that are contained on the compact disc (submitted herewith) in the file named “DF02 PCT (SEQL)” (which is 7 kilobytes in size and was created on 10 Feb. 2009) are filed herewith and are incorporated herein by reference in their entirety.

### BACKGROUND

Class D cyclins are regulators of G1-S phase transition in cell cycle progression. Class D cyclins control the progression through the restriction point during late-G1 phase, when cells lose their dependency on mitogens and commit to DNA synthesis. Class D cyclins, which include cyclin D1, cyclin D2, and cyclin D3, bind to and form complexes with cyclin-dependent kinase 4 and cyclin-dependent kinase 6. These complexes phosphorylate, and thereby inactivate, retinoblastoma protein (pRB). The phosphorylation (and associated inactivation) of pRB leads to the activation of various genes associated with progression of late G1 and S phases. (Tashiro et al. (2007) Cancer Sci 95:629-635; Caldon et al. (2006) 97:261-274; Fu et al. (2004) Endocrinology 145:5439-5447.)

Cyclin D1 levels are increased in many tumors and elevated cyclin D1 levels have been associated with increased tumor growth. For example, elevated levels of cyclin D1 are observed in over 50% of breast cancers; many of these breast cancers are also estrogen receptor (ER)-positive breast cancers. Additionally, cyclin D1 is associated with estrogen-induced breast cancer and reports have implicated cyclin D1 as having a critical role in human breast cancer cell-cycle control. (Roy et al. (2006) The Breast 15:718-727; Masood et al. (1992) Diagn Cytopathol 8:475-91.)

Endocrine therapy, such as, for example, the selective estrogen receptor modulator (SERM) tamoxifen, is commonly used to treat breast cancer. However, only 50%-60% of ER-positive breast cancer patients respond to tamoxifen therapy. Moreover, certain patients with localized breast cancer, and all patients with metastatic breast cancer, become resistant to SERM therapies. (See Baum M. (1998) Br J Cancer 78:S1-4; Massarweh et al. (2006) Endocr Relat Cancer 13:S15-24.) Elevated levels of cyclin D1 provide a growth advantage to breast tumor cells as well as provide resistance to endocrine therapy. (Musgrove et al. (1994) PNAS 91:8022-6; Kenny et al. (1999) Clin Cancer Res 5:2069-2076.)

Current treatments for various cancers and other disorders associated with elevated cyclin D levels are limited, and improved methods for treating such disorders would be beneficial. The present invention provides methods for decreasing the level or activity of cyclin D, thereby delaying or preventing tumor growth and proliferation in subjects having cancer or other disorders associated with elevated cyclin D levels.

### SUMMARY OF THE INVENTION

The present invention provides a method for identifying a modulator of cyclin D levels or activity, the method comprising: (a) measuring the activity of a prolyl hydroxylase in the presence and in the absence of a candidate modulator under conditions suitable for the prolyl hydroxylase to hydroxylate a polypeptide substrate in the absence of the candidate modulator; (b) comparing the activity of a prolyl hydroxylase measured in the presence and in the absence of a candidate modulator in step (a); and (c) identifying the candidate modulator as a modulator of cyclin D levels if the activity of the prolyl hydroxylase differs in the presence and in the absence of the candidate modulator. In some embodiments, the cyclin D is cyclin D1, cyclin D2, or cyclin D3. In other embodiments, the modulation of cyclin D levels is a decrease in cyclin D gene expression or cyclin D protein levels. In another embodiment, the prolyl hydroxylase is a hypoxia-inducible factor (HIF) prolyl hydroxylase. In yet another embodiment, the prolyl hydroxylase is EGLN2. In certain embodiments, the activity or expression of the prolyl hydroxylase is measured in a cell. In other embodiments, the activity or expression of the prolyl hydroxylase is measured in vitro. In some embodiments, the cyclin D or the prolyl hydroxylase or both are recombinantly expressed by the cell expressing cyclin D and the prolyl hydroxylase.

In another embodiment, the present invention provides a method for identifying a modulator of cyclin D levels, the method comprising: (a) measuring the activity of a prolyl hydroxylase in the presence and in the absence of a candidate modulator under conditions suitable for the prolyl hydroxylase to hydroxylate a polypeptide substrate in the absence of the candidate modulator; (b) comparing the activity of a prolyl hydroxylase measured in the presence and in the absence of a candidate modulator in step (a); (c) identifying the candidate modulator as one that alters the activity of the prolyl hydroxylase if the activity of the prolyl hydroxylase differs in the presence and in the absence of the candidate modulator; (d) measuring the levels of cyclin D in the presence and in the absence of the candidate modulator identified in step (c); (e) comparing the levels of cyclin D measured in the presence and in the absence of the candidate modulator in step (d); and (f) identifying the candidate modulator as a modulator of cyclin D levels if the level of cyclin D differs in the presence and in the absence of the candidate modulator. In some embodiments, the cyclin D is cyclin D1, cyclin D2, or cyclin D3. In other embodiments, the modulation of cyclin D levels is a decrease in cyclin D gene expression or cyclin D protein levels. In another embodiment, the prolyl hydroxylase is a hypoxia-inducible factor (HIF) prolyl hydroxylase. In yet another embodiment, the prolyl hydroxylase is EGLN2. In certain embodiments, the activity or expression of the prolyl hydroxylase is measured in a cell. In other embodiments, the activity or expression of the prolyl hydroxylase is measured in vitro. In another embodiment, the activity or expression of the prolyl hydroxylase is measured in situ. In some embodiments, the cyclin D or the prolyl hydroxylase or both are recombinantly expressed by the cell expressing cyclin D and the prolyl hydroxylase.

In another embodiment, the present invention provides a method for identifying a modulator of EGLN2 expression or activity, the method comprising: (a) measuring the levels of cyclin D in the presence and in the absence of a candidate modulator; (b) comparing the levels of cyclin D measured in the presence and in the absence of a candidate modulator in step (a); and (c) identifying the candidate modulator as a modulator of EGLN2 expression or activity if the levels of cyclin D differs in the presence and in the absence of the candidate modulator. In some embodiments, the modulation of cyclin D levels is a decrease in cyclin D gene expression or cyclin D protein levels. In certain embodiments, the levels of cyclin D are measured in a cell. In other embodiments, the levels of cyclin D are measured in vitro. In another embodiment, the levels of cyclin D are measured in situ. In some embodiments, the cyclin D or EGLN2 is recombinantly expressed by the cell expressing cyclin D or EGLN2. In particular embodiments, the cyclin D is cyclin D1, cyclin D2, or cyclin D3.

In certain embodiments, the activity or expression of the prolyl hydroxylase is measured by measuring the hydroxylation of a polypeptide substrate. In some embodiments, the polypeptide substrate is an alpha subunit of hypoxia-inducible factor (HIF-α) or a fragment of HIF-α containing a proline residue.

In another embodiment, the present invention provides methods for producing or manufacturing a pharmaceutical composition or medicament, the method comprising: (a) identifying a modulator of cyclin D levels or activity according to the methods described above; and (b) combining the modulator with a pharmaceutically acceptable carrier. In one embodiment, the cyclin D is cyclin D1, cyclin D2, or cyclin D3.

In other embodiments, the present invention provides a method for decreasing the level of cyclin D in a cell, the method comprising exposing the cell to an agent that inhibits the activity or expression of a prolyl hydroxylase. In one embodiment, the prolyl hydroxylase is EGLN2. In another embodiment, the cyclin D is cyclin D1, cyclin D2, or cyclin D3. In yet another embodiment, the present invention provides a method for decreasing cyclin D protein levels or cyclin D mRNA levels in a subject, the method comprising administering to the subject an effective amount of an agent that inhibits the activity or expression of a prolyl hydroxylase, thereby decreasing cyclin D protein levels or cyclin D mRNA levels in the subject. In one aspect, the prolyl hydroxylase is a hypoxia-inducible factor (HIF) prolyl hydroxylase. In another aspect, the prolyl hydroxylase is EGLN2. In other embodiments of the present aspect, the cyclin D is cyclin D1, cyclin D2, or cyclin D3.

Methods for treating a disorder associated with elevated cyclin D levels or activity in a subject are also provided by the present invention. In one embodiment, the present invention provides a method for treating a disorder associated with elevated cyclin D levels or activity in a subject, the method comprising administering to the subject an effective amount of an agent that inhibits the activity or expression of a prolyl hydroxylase. In certain embodiments, the prolyl hydroxylase is EGLN2. In other embodiments, the cyclin D is cyclin D1, cyclin D2, or cyclin D3. In other embodiments, the disorder associated with elevated cyclin D levels or activity is cancer. In yet other embodiments, the disorder associated with elevated cyclin D levels or activity is an estrogen-receptor positive cancer, an estrogen-dependent cancer, or a cancer resistant to endocrine therapy, such as, for example, a tamoxifen-resistant cancer.

In another embodiment, the present invention provides a method for treating or preventing cancer associated with elevated levels or activity of cyclin D in a subject, the method comprising administering to the subject an effective amount of an agent that inhibits the activity or expression of a prolyl hydroxylase. In some embodiments, the cancer is an estrogen-receptor positive cancer, an estrogen-dependent cancer, or a cancer resistant to endocrine therapy, such as, for example, a tamoxifen-resistant cancer. In certain embodiments, the prolyl hydroxylase is EGLN2. In another embodiment, the cyclin D is cyclin D1, cyclin D2, or cyclin D3. In other embodiments, the present invention provides a method for decreasing tumor formation or tumor weight associated with elevated levels or activity of cyclin D in a subject, the method comprising administering to the subject an effective amount of an agent that inhibits the activity or expression of a prolyl hydroxylase. In another embodiment, the cyclin D is cyclin D1, cyclin D2, or cyclin D3. In yet another embodiment, the present invention provides a method for decreasing cell proliferation associated with elevated levels or activity of cyclin D in a subject, the method comprising administering to the subject an effective amount of an agent that inhibits the activity or expression of a prolyl hydroxylase. In another embodiment, the cyclin D is cyclin D1, cyclin D2, or cyclin D3.

In various embodiments, the agent used in the present methods is a compound. In some embodiments, the compound is a structural mimetic of 2-oxoglutarate, wherein the compound inhibits the target prolyl hydroxylase enzyme competitively with respect to 2-oxoglutarate and noncompetitively with respect to iron. In other embodiments, compounds useful for the methods of the present invention include variously substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo-4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, and 4-hydroxy-pyrrolopyridine-5-carbonyl-glycines.

Pharmaceutical compositions or medicaments effective for use in any of the present methods are provided herein. In various embodiments, the compositions or medicaments comprise an effective amount of an agent that inhibits the activity or expression of a prolyl hydroxylase and an acceptable carrier. In one aspect, the prolyl hydroxylase is a hypoxia-inducible factor (HIF) prolyl hydroxylase. In another aspect, the prolyl hydroxylase is EGLN2.

In another embodiment, the agent used in the methods of the present invention is short interfering RNA (siRNA), wherein the siRNA inhibits the target prolyl hydroxylase activity or expression. In one embodiment, the siRNA comprises a nucleotide sequence substantially identical to a target sequence of about 19 to about 25 contiguous nucleotides in EGLN2 mRNA. In particular embodiments, the siRNA comprises the nucleotide sequence of SEQ ID NO:1 or SEQ ID NO:2. In another embodiment, the agent used in the methods of the present invention is short hairpin RNA (shRNA), wherein the shRNA inhibits the target prolyl hydroxylase activity or expression. In particular embodiments, the shRNA comprises the nucleotide sequence of SEQ ID NO:1 or SEQ ID NO:2.

In some embodiments, the present invention provides a method for reducing prolyl hydroxylase activity or expression in a subject, the method comprising administering to the subject a siRNA or a shRNA comprising the nucleotide sequence of SEQ ID NO:1 or SEQ ID NO:2, thereby reducing prolyl hydroxylase activity or expression in the subject. In some embodiments, the prolyl hydroxylase is EGLN2. In other embodiments, the present invention provides a method for reducing cyclin D levels or activity in a subject, the method comprising administering to the subject a siRNA or a shRNA comprising the nucleotide sequence of SEQ ID NO:1 or SEQ ID NO:2, thereby reducing cyclin D levels or activity in the subject. In some embodiments, the cyclin D is selected from the group consisting of cyclin D1, cyclin D2, and cyclin D3. In certain aspects, the cyclin D levels are cyclin D protein levels or cyclin D mRNA levels.

Methods are also provided by the present invention for treating a disorder associated with elevated levels or activity of cyclin D, the methods comprising administering to a subject having or at risk for having a disorder associated with elevated levels or activity of cyclin D a siRNA or a shRNA comprising the nucleotide sequence of SEQ ID NO:1 or SEQ ID NO:2, thereby treating the disorder associated with elevated levels or activity of cyclin D. In some embodiments, the cyclin D is selected from the group consisting of cyclin D1, cyclin D2, and cyclin D3. In other embodiments, the disorder associated with elevated levels or activity of cyclin D is cancer. In yet other embodiments, the disorder associated with elevated levels or activity of cyclin D is an estrogen-receptor positive cancer, an estrogen-dependent cancer, or a cancer resistant to endocrine therapy, such as, for example, a tamoxifen-resistant cancer.

These and other embodiments of the present invention will readily occur to those of skill in the art in light of the disclosure herein, and all such embodiments are specifically contemplated

### BRIEF DESCRIPTION OF THE DRAWINGS

FIG. 1 sets forth data showing agents and methods of the present invention reduced cyclin D1 protein levels in human osteosarcoma cells.

FIG. 2 sets forth data showing agents and methods of the present invention reduced cyclin D1 protein levels in human breast cancer cells.

FIG. 3 sets forth data showing agents and methods of the present invention reduced cyclin D1 protein levels in human osteosarcoma cells and human cervical adenocarcinoma cells.

FIG. 4 sets forth data showing agents and methods of the present invention reduced cyclin D1 protein levels in human cervical adenocarcinoma cells.

FIGS. 5A and 5B set forth data showing agents and methods of the present invention reduced cyclin D1 mRNA levels in human osteosarcoma cells and human cervical adenocarcinoma cells.

FIG. 6 sets forth data showing EGLN2 increased cyclin D1 promoter activity in human osteosarcoma cells.

FIGS. 7A, 7B, and 7C set forth data showing agents and methods of the present invention decreased estrogen-induced increases in cyclin D1 mRNA and protein levels in human breast carcinoma cells.

FIG. 8 sets forth data showing agents and methods of the present invention decreased cyclin D1 protein levels in human breast carcinoma cells.

FIG. 9 sets forth data showing agents and methods of the present invention decreased cyclin D1 protein levels in human breast carcinoma cells.

FIG. 10 sets forth data showing agents and methods of the present invention reduced proliferation of human breast carcinoma cells.

FIG. 11 sets forth data showing agents and methods of the present invention reduced proliferation of estrogen-dependent human breast carcinoma cells.

FIG. 12 sets forth data showing agents and methods of the present invention reduced proliferation of estrogen-dependent human breast carcinoma cells.

FIG. 13 sets forth data showing agents and methods of the present invention reduced cyclin D1 protein levels in human breast carcinoma cells.

FIGS. 14A and 14B set forth data showing methods and agents of the present invention reduced tumor formation in vivo.

FIG. 15 sets forth data showing methods and agents of the present in invention reduced tumor formation in vivo.

FIG. 16 sets forth data showing methods and agents of the present invention reduced tumor weight of breast tumors in vivo.

FIG. 17 sets forth data showing methods and agents of the present invention reduced cyclin D1 protein levels in breast tumors in vivo.

### DETAILED DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments; a reference to a “compound” may be a reference to one or more compounds and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

The section headings are used herein for organizational purposes only, and are not to be construed as in any way limiting the subject matter described herein.

### Methods

Methods and agents of the present invention decreased cyclin D1 protein levels in cells. (See e.g., Example 1.) Additionally, methods and agents of the present invention decreased cyclin D1 mRNA levels in cells. (See e.g., Example 3.) Thus, the present invention provides methods for decreasing or reducing the level or activity of cyclin D in a cell by administering to the cell an effective amount of an agent that inhibits EGLN2 expression or activity. In one aspect, the administering is ex vivo. In another aspect, the administering is in vivo. In one embodiment, the methods of the present invention decrease or reduce the level of cyclin D protein in a cell by inhibiting EGLN2 expression or activity. In another embodiment, the methods of the present invention decrease or reduce cyclin D mRNA levels in a cell by inhibiting EGLN2 expression or activity. In various embodiments, the cyclin D is cyclin D1, cyclin D2, or cyclin D3.

Various disorders, such as cancer, are associated with elevated cyclin D levels or activity. For example, elevated cyclin D levels or activity are associated with increased tumor growth and proliferation. Methods and agents of the present invention reduced proliferation of breast carcinoma cells. (See, e.g., Example 6.) Additionally, methods and agents of the present invention decreased tumor formation and tumor weight in vivo. (See, e.g., Example 8.) Thus, the present invention provides methods for treating disorders associated with elevated levels or activity of cyclin D. In one embodiment, the present invention provides a method for treating a disorder associated with elevated levels or activity of cyclin D in a subject, the method comprising administering to the subject an effective amount of an agent that inhibits EGLN2 expression or activity, thereby treating the disorder associated with elevated levels or activity of cyclin D. In some aspects, the disorder associated with elevated levels or activity of cyclin D is cancer. In other aspects, the disorder associated with elevated levels or activity of cyclin D is breast cancer. In various embodiments, the cyclin D is cyclin D1, cyclin D2, or cyclin D3.

In one embodiment, the present invention provides a method for treating an estrogen receptor (ER)-positive cancer in a subject, the method comprising decreasing or reducing the level of cyclin D in the subject by administering to the subject an effective amount of an agent that inhibits EGLN2 expression or activity, thereby treating the ER-positive cancer in the subject. In various embodiments, the cyclin D is cyclin D1, cyclin D2, or cyclin D3.

Methods and agents of the present invention decreased tumor formation of estrogen-dependent cancer cells in vivo. (See, e.g., Example 8.) Therefore, in another embodiment, the present invention provides a method for treating an estrogen-dependent cancer in a subject, the method comprising decreasing or reducing the level of cyclin D in the subject by administering to the subject an effective amount of an agent that inhibits EGLN2 expression or activity, thereby treating the estrogen-dependent cancer in the subject. In various embodiments, the cyclin D is cyclin D1, cyclin D2, or cyclin D3.

In yet another embodiment, the present invention provides a method for treating a cancer resistant to endocrine therapy in a subject, the method comprising decreasing or reducing the level of cyclin D in the subject by administering an effective amount of an agent that inhibits EGLN2 expression or activity, thereby treating the cancer resistant to endocrine therapy in the subject. In various embodiments, the cyclin D is cyclin D1, cyclin D2, or cyclin D3.

### Agents

The terms “inhibitor of EGLN2 enzyme activity”, “inhibits EGLN2 expression or activity”, and “EGLN2 inhibitor”, and as abbreviated the term “inhibitor”, are used interchangeably and refer to any agent that reduces the expression or an activity of the EGLN2 enzyme. For example, for purposes of measuring EGLN2 activity, the activity of EGLN2 on hydroxylation of one or more proline residues on the alpha subunit of hypoxia-inducible factor (HIFα), or on fragments thereof, may be measured in the presence and absence of a test agent (e.g., candidate modulator). A decrease in the extent of hydroxylation of HIFα, or of fragments thereof, when test agent is present compared to the extent of hydroxylation of HIFα when test agent is absent would indicate the test agent (e.g., candidate modulator) is an inhibitor of EGLN2 activity (i.e., an EGLN2 inhibitor) for purposes of the present invention.

In some aspects, an inhibitor of EGLN2 activity is a small molecule. In one aspect, the inhibitor of EGLN2 activity is an inhibitor of succinate dehydrogenase (SDH) activity and may be selected from the group consisting of, but not limited to, malonic acid, 3-nitroproprionic acid, and theonyl trifluoracetone. EGLN2 enzyme activity is generally affected by SDH activity due to feedback inhibition of EGLN2 by succinate, a compound that is converted to fumarate by SDH.

In other aspects, the inhibitor of EGLN2 activity is a structural mimetic of 2-oxoglutarate. In certain aspects, the structural mimetic of 2-oxoglutarate (i.e., a 2-oxoglutarate mimetic) is selected from the group consisting of dimethyloxalylglycine, N-oxalylglycine, N-oxalyl-2S-alanine, and N-oxalyl-2R-alanine. Additional compounds that may inhibit EGLN2 enzyme activity are described in, e.g., Majamaa et al. (1984) Eur J Biochem 138:239-245; Majamaa et al. (1985) Biochem J 229:127-133; Kivirikko, and Myllyharju (1998) Matrix Biol 16:357-368; Bickel et al. (1998) Hepatology 28:404-411; Friedman et al. (2000) Proc Natl Acad Sci USA 97:4736-4741; Franklin (1991) Biochem Soc Trans 19:812-815; and Franklin et al. (2001) Biochem J 353:333-338. Additionally, compounds that inhibit EGLN2 activity have been described in, e.g., International Publication Nos. WO 03/049686, WO 02/074981, WO 03/080566, WO 2004/108681, WO 2006/094292, WO 2007/038571, WO 2007/090068, WO 2007/070359, WO 2007/103905, and WO 2007/115315. Compounds for use in the present methods inhibit EGLN2 activity, and may additionally inhibit activity of related enzymes, e.g., FIH, etc. Preferred compounds selectively inhibit EGLN2, i.e., show greater inhibition of EGLN2 than inhibition of related enzymes. The inhibitor of EGLN2 activity can be administered alone or in combination with another agent for treating a disorder such as cancer. An exemplary compound for use in the present invention is 4-oxo-1,4-dihydro-[1,10]phenanthroline-3-carboxylic acid (Compound A).

Over-expression (e.g., expression above normal levels) of EGLN2 is associated with increased levels of cyclin D1. The examples provided herein demonstrate that reducing EGLN2 (GenBank Accession No. AAH36051; GI:23273572; PHD1, HPH3) activity or expression reduces cyclin D1 (GenBank Accession No. NP444284; GI:16950655) levels in various cells, including cells derived from cancerous tissues. The examples provided herein also show that the known prolyl hydroxylase catalytic (i.e., enzyme) activity of EGLN2 is required for cyclin D1 regulation, and inhibition of EGLN2 by various agents including, for example, desferrioxamine, a phenanthroline compound, and dimethyl-oxalylglycine, result in decreased or reduced cyclin D1 levels. Other studies described herein show that knockdown of EGLN2 expression and activity with siRNA or shRNA also decreased cyclin D1 levels.

The present invention provides a method for identifying a modulator of cyclin D levels, the method comprising: (a) measuring the activity of a prolyl hydroxylase in the presence and in the absence of a candidate modulator under conditions suitable for the prolyl hydroxylase to hydroxylate a polypeptide substrate in the absence of the candidate modulator; (b) comparing the activity of a prolyl hydroxylase measured in the presence and in the absence of a candidate modulator in step (a); (c) identifying the candidate modulator as one that alters the activity of the prolyl hydroxylase if the activity of the prolyl hydroxylase differs in the presence and in the absence of the candidate modulator; (d) measuring the levels of cyclin D in the presence and in the absence of the candidate modulator identified in step (c); (e) comparing the levels of cyclin D measured in the presence and in the absence of the candidate modulator in step (d); and (f) identifying the candidate modulator as a modulator of cyclin D levels if the level of cyclin D differs in the presence and in the absence of the candidate modulator. In one aspect, the cyclin D is cyclin D1 (GenBank Accession No. NP444284; GI:16950655). In another aspect, the cyclin D is cyclin D2 (GenBank Accession No. NP001750; GI:4502617). In yet another aspect, the cyclin D is cyclin D3 (GenBank Accession No. NP001751; GI:4502619). In some aspects, the prolyl hydroxylase is EGLN2. In certain aspects, the activity or expression of the prolyl hydroxylase is measured by determining the hydroxylation of a polypeptide substrate, such as, for example, the alpha subunit of HIF or any fragment thereof containing a proline residue.

The invention further provides a method for identifying a modulator of EGLN2 expression or activity, the method comprising: (a) measuring the levels of cyclin D in the presence and in the absence of a candidate modulator; (b) comparing the levels of cyclin D measured in the presence and in the absence of a candidate modulator in step (a); and (c) identifying the candidate modulator as a modulator of EGLN2 expression or activity if the levels of cyclin D differs in the presence and in the absence of the candidate modulator. In some embodiments, the modulation of cyclin D levels is a decrease in cyclin D gene expression or cyclin D protein levels. In certain embodiments, the levels of cyclin D are measured in a cell. In other embodiments, the levels of cyclin D are measured in vitro. In another embodiment, the levels of cyclin D are measured in situ. In some embodiments, the cyclin D or EGLN2 is recombinantly expressed by the cell expressing cyclin D or EGLN2. In one aspect, the cyclin D is cyclin D1 (GenBank Accession No. NP444284; GI:16950655). In another aspect, the cyclin D is cyclin D2 (GenBank Accession No. NP001750; GI:4502617). In yet another aspect, the cyclin D is cyclin D3 (GenBank Accession No. NP001751; GI:4502619).

The invention also provides a method for manufacturing a pharmaceutical composition, the method comprising: (a) identifying a modulator of cyclin D according to the methods described herein; and (b) combining the modulator with a pharmaceutically acceptable carrier. In one aspect, the cyclin D is cyclin D1. In another aspect, the cyclin D is cyclin D2. In yet another aspect, the cyclin D is cyclin D3.

### Subjects

The invention is applicable to a variety of different organisms, including for example, vertebrates, large animals, and primates. In some embodiments, the subject is a cell, an organism, or an organ. In certain embodiments, the subject is a mammalian subject; in particular embodiments, the subject is a human subject. Although medical applications with humans are clearly foreseen, veterinary applications are also envisaged herein.

It is contemplated in specific embodiments of the present invention that the present methods are directed at decreasing or reducing cyclin D1 levels or activity in a subject in need thereof, wherein the subject has or is at risk for having elevated cyclin D1 levels or activity. In some embodiments, the subject has or is at risk for having a disorder associated with elevated or increased cyclin D levels or activity. In certain embodiments, the subject has or is at risk for having cancer. In other embodiments, the subject has or is at risk for having breast cancer, an estrogen receptor positive cancer, or an estrogen-dependent cancer. In yet other embodiments, the subject has or is at risk for having cancer, wherein the cancer is resistant to endocrine therapy. In other embodiments, the subject has or is at risk for having a cancer resistant to a selective estrogen receptor modulator (SERM) therapy, including, for example, tamoxifen.

In certain embodiments of the present invention, the cancer may in particular be cancer of the lung, colon, prostate, esophagus, bladder, skin, liver, blood, head, neck, thyroid, or breast. However, other cancers are also envisaged in the methods of the present invention. For example, the cancer may be ovarian cancer, including advanced ovarian cancer. Stage I, II, III, or IV cancer may be treated according to the present invention. In certain embodiments, the breast cancer is classified as estrogen-receptor (ER) positive or estrogen-dependent cancer. Such cancers are associated with elevated levels of cyclin D1 (i.e. cyclin D1 levels above normal levels), which provides a growth advantage to breast tumor cells and resistance to endocrine therapy, including resistance to selective estrogen receptor modulators (SERM) (e.g., tamoxifen).

Methods for identifying subjects with elevated cyclin D levels or expression are well-known and available to one of skill in the art. For example, in the clinic, subjects with elevated cyclin D levels are identified by immunostaining of biopsy tissue for cyclin D1. (See, e.g., Arber et al. (1996) Gastroenterology. 110:669-74.) Breast cancer subjects with elevated cyclin D1 levels are also identified using such techniques. (See, e.g., Rudas et al. (2008) Clin Cancer Res. 14:1767-74.) Additionally, fluorescence in-situ hybridization (i.e. FISH) is used to evaluate cyclin D1 gene (CCND1) amplification in biopsy from subjects with cancer. (See, e.g., Jirström et al. (2005) Cancer Res. 65:8009-16.) These techniques, and others available to one of skill in the art, may be employed to identify a subject as a subject with elevated cyclin D levels or expression.

Methods for identifying subjects with estrogen-receptor positive cancer or estrogen dependent cancer are well-known and available to one of skill in the art. For example, Sannino et al. provides a method for assessing estrogen-receptor status of cancer patients in routinely processed tumor samples ((1994) J Clin Pathol. 47:90-2). Similarly, Poulin et al. describes methods for identifying estrogen-dependent breast cancer cells ((1989) Breast Cancer Res Treat. 13:265-76). These techniques, and others available to one of skill in the art, may be utilized to identify a subject as a subject with estrogen-receptor positive cancer or estrogen dependent cancer.

It is also contemplated that the cancer is associated with formation of solid tumors, including carcinomas, such as adenocarcinomas and epithelial carcinomas. Such cancers can include, but are not limited to, lung cancer, including non-small cell lung cancer, and large cell carcinoma types, as well as small cell lung cancer; colon cancer, including colon metastasized to liver and including colorectal cancers; breast cancer; and ovarian cancer, as mentioned above. Cancers that can be associated with solid tumors further include, but are not limited to, kidney or renal cancers, including, for example, renal cell carcinomas; cancer of the bladder; liver cancer, including, for example, hepatocellular carcinomas; cancer of the gastrointestinal tract, including rectal, esophageal, pancreatic, and stomach cancer; gynecological cancers, including cervical, uterine, and endometrial cancers; prostate cancer or testicular cancer; nasopharyngeal cancer; thyroid cancer, for example, thyroid papillary carcinoma; cancer of the head, neck, or brain; nervous system cancers, including neuroblastomas. Carcinomas include, but are not limited to, adenocarcinomas and epithelial carcinomas.

### Inhibitors of EGLN2

The present invention provides various inhibitors of EGLN2 which are effective at decreasing or reducing cyclin D1 levels. Small molecule compounds which may be used in the present methods include 2-oxoglutarate analogs including, but not limited to, dimethyloxalylglycine, N-oxalylglycine, N-oxalyl-2S-alanine, N-oxalyl-2R-alanine, an enantiomer of N-oxalyl-2S-alanine. In particular embodiments, the small molecule compound useful in the present methods is 4-Oxo-1,4-dihydro-[1,10]phenanthroline-3-carboxylic acid (Compound A). Other N-oxalyl-amino acid compounds are among the potentially useful inhibitors.

Additional compounds that may be used to inhibit EGLN2 are described in, e.g., Majamaa et al. (1984) Eur J Biochem 138:239-245; Majamaa et al. (1985) Biochem J 229:127-133; Kivirikko, and Myllyharju (1998) Matrix Biol 16:357-368; Bickel et al. (1998) Hepatology 28:404-411; Friedman et al. (2000) Proc Natl Acad Sci USA 97:4736-4741; Franklin (1991) Biochem Soc Trans 19):812-815; and Franklin et al. (2001) Biochem J 353:333-338. Additionally, compounds that inhibit EGLN2 have been described in, e.g., International Publication Nos. WO 03/049686, WO 02/074981, WO 03/080566, WO 2004/108681, WO 2006/094292, WO 2007/038571, WO 2007/090068, WO 2007/070359, WO 2007/103905, and WO 2007/115315.

Additional inhibitors of EGLN2 expression or activity may be identified using various methods known to those of skill in the art. For example, a screening assay as described in International Publication No. WO 2005/118836 may be used to screen compounds for selective activity against EGLN2. Compounds which may be screened using the assay may be natural or synthetic chemical compounds. Extracts of plants, microbes, or other organisms, which contain several characterized or uncharacterized components may also be used. Combinatorial libraries (including solid phase synthesis and parallel synthesis methodologies) provide an efficient way of testing larges numbers of different substances for the ability to modulate hydroxylation. Further, the compounds described above can be similarly tested in various assays to identify those having particular selectivity for EGLN2. Such compounds are particularly advantageous in the present methods to reduce potential undesirable side effects.

Other inhibitors of EGLN2 include short interfering RNA (siRNA). Short interfering RNAs can comprise a duplex, or double-stranded region, of about 19 to about 25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. Short interfering RNAs can also contain additional sequences; non-limiting examples of such sequences include linking sequences, or loops, as well as stem and other folded structures.

Short interfering RNA agents (e.g. oligonucleotides) directed against EGLN2 may be used to inhibit EGLN2 expression or activity. Any siRNA useful for the methods of the present invention can be selected from any stretch of about 19 to about 25 contiguous nucleotides in the EGLN2 mRNA sequences. For example, the human cDNA sequence for EGLN2 (SEQ ID NO:11) may be used to select siRNA useful for the methods of the present invention. In particular embodiments, the siRNA useful in the present methods comprises the nucleotide sequence of SEQ ID NO: 1 or SEQ ID NO:2. Techniques for selecting sequences for siRNA are well-known in the art. (See, e.g., Tuschl et al. (2002) The siRNA User Guide, incorporated in its entirely by reference herein.)

Short interfering RNAs useful in the present methods can be obtained using a number of techniques known to those of skill in the art. For example, the siRNA can be chemically synthesized or recombinantly produced using methods known in the art. (See, e.g., the Drosophila in vitro system described in U.S. Patent Application Publication Nos. US2002/0086356 and US2005/0080031, and International Publication No. WO 03/070881, each of which is incorporated by reference herein in its entirety.)

Short hairpin RNA (shRNA) is a sequence of RNA that makes a tight hairpin turn that can be used to silence gene expression by RNA interference. (See, e.g., Paddison et al. (2002) Genes & Dev. 16:948-958.) shRNA agents can be administered using a vector introduced into cells and utilizes a promoter, such as, for example, the U6 promoter. The shRNA hairpin structure is cleaved by the cellular machinery into siRNA, which is then bound to the RNA-induced silencing complex (RISC). This complex binds to and cleaves mRNAs which match the siRNA that is bound to it. Techniques for constructing shRNA expression vectors are known in the art. (See, e.g., McIntyre et al. (2006) BMC Biotechnol. 6:1.) The human cDNA sequence for EGLN2 (SEQ ID NO:11) may, for example, be used to select shRNA useful for the methods of the present invention. In particular embodiments, the shRNA useful in the present methods comprises the nucleotide sequence of SEQ ID NO:1 or SEQ ID NO:2.

### Measuring Hydroxylation by Measuring VHL Binding

The present invention provides various modulators of EGLN2 which are effective at decreasing or reducing cyclin D levels or expression. Modulators of EGLN2 can be identified using various assays, including for example, assays that measure hydroxylation of HIF-1α. In certain circumstances, it can be useful to measure the binding of VHL to HIF-1α as a measure of hydroxylation, for example, to detect or quantify hydroxylated HIF-1α. The VHL is preferably human VHL (GenBank® Accession Numbers AF010238 and L15409). Other mammalian VHL (e.g., mouse: GenBank Accession number U12570; rat: GenBank Accession numbers U 14746 and S80345; or C. elegans VHL (GenBank Accession number F08G12.4) might be useful in some circumstances. It may be possible to use a variant VHL or fragment of VHL that retains the ability to interact directly with a hydroxylated HIF-1α. The ability of VHL fragments and variants to bind to a HIF-1α may be tested as described below.

VHL gene sequences may also be obtained by routine cloning techniques. A wide variety of techniques are available for this, for example, PCR amplification and cloning of the gene using a suitable source of mRNA (e.g., from an embryo or a liver cell), obtaining a cDNA library from a mammalian, vertebrate, invertebrate or fungal source, e.g., a cDNA library from one of the above-mentioned sources, probing the library with a polynucleotide of the invention under stringent conditions, and recovering a cDNA encoding all or part of the VHL protein of that mammal. It is not necessary to use the entire VHL protein in the assay (including their mutants and other variants). Fragments of the VHL may be used, provided such fragments retain the ability to interact with the target domain of the HIF-1α. Generally fragments will be at least 40, preferably at least 50, 60, 70, 80, or 100 amino acids in size.

Fragments of the HIF-1α may be used, provided that the fragments retain the ability to interact with a wild-type VHL, preferably wild-type human VHL. Such fragments are desirably at least 20, preferably at least 40, 50, 75, 100, 200, 250, or 300 amino acid residues in size. The fragment retains the proline hydroxylation site.

The amount of VHL and HIF-1α may be varied depending upon the scale of the assay. In general, relatively equimolar amounts of the two components are used.

Where assays of the invention are performed within cells, the cells may be treated to provide or enhance a normoxic environment, i.e., an oxygen level similar to that found in normal air at sea level. As a control cells may also be cultured under hypoxic conditions, e.g., oxygen levels at 0.1 to 1.0%. The cells may also be treated with compounds which mimic hypoxia and cause up regulation of HIF-1α. Such compounds include iron chelators (desferrioxamine, O-phenanthroline or hydroxypyridinones (e.g. 1,2-diethyl hydroxypyridinone (CP94) or 1,2-dimethyl hydroxypyridinone (CP20)), cobalt (II), nickel (II) or manganese (II)). For cell based assays the proteins may be expressed eukaryotic cells, such as yeast, insect, mammalian, primate, and human cells.

### Assays for Identifying Modulators of Cyclin D Expression

The present invention shows that agents that decrease or reduce the expression or activity of EGLN2 decrease or reduce the levels of cyclin D1; therefore, modulators of cyclin D1 levels can be identified by identifying modulators of EGLN2 expression or activity, as described below. These modulators can be optionally tested for their ability to modulate levels of cyclin D1 in a cell.

### Assays for Identifying Modulators of EGLN2 Expression or Activity

The present invention shows that agents that decrease or reduce the levels of cyclin D1 decrease the expression or activity of EGLN2. (See, e.g., Example 2.) Thus, agents that modulate the expression or activity of EGLN2 can be identified using an assay of cyclin D1 levels. An agent which modulates the expression or activity of EGLN2 can be identified by a method comprising: (a) measuring the levels of cyclin D1 in the presence and in the absence of a candidate modulator; (b) comparing the levels of cyclin D1 measured in the presence and in the absence of a candidate modulator in step (a); and (c) identifying the candidate modulator as a modulator of EGLN2 expression or activity if the levels of cyclin D1 differs in the presence and in the absence of the candidate modulator. The levels of cyclin D1 may be determined by measuring the cyclin D1 protein levels (e.g., using immunoblot analysis) or the levels of cyclin D1 expression (e.g., using fluorescent in-situ hybridization) in a cell. The levels of cyclin D1 can also be measured in vitro, in a tissue, in an organ, in a tumor, or in any sample obtained from a subject (e.g., biopsy). Assays for cyclin D1 levels are known in the art (see, e.g., Arber et al. (1996) Gastroenterology. 110:669-74; Rudas et al. (2008) Clin Cancer Res. 14:1767-74; and Jirström et al. (2005) Cancer Res. 65:8009-16) and described herein. (See, e.g., Example 1.)

In an exemplary assay for cyclin D1 levels, whole cell extracts are prepared and western blot analysis are performed as follows. Cell lysates are prepared by suspending 1 10⁶ cells in 50 μL lysis buffer (20 mM 4-(2-hydroxyethyl)-1-piperazineethanesulfonic acid (pH 7.9), 350 mM NaCl, 1 mM MgCl2, 0.5 mM EDTA, 0.1 mM thyleneglycotetraacetic acid, 1% Nonidet P-40, 0.5 mM dithiothreitol, 0.4 mM 4-(2-aminoethyl)-benzenesulfonyl fluoride hydrochloride) for 20 minutes at 4° C. After centrifugation at 10,000 g for 20 minutes, the supernatant is used as a whole-cell extract. Protein concentration of the whole-cell extract is measured using a Quick Start Bradford Dye Reagent (Bio-Rad Laboratories). For Western blot analysis, 50 μg of whole-cell extracts is separated on NuPAGE 12% Bis-Tris Gel (Invitrogen) and transferred to a Hybond-P membrane (Amersham Biosciences). After washing the membrane with Tris-buffered saline containing 0.1% Tween 20 (TBST), the membrane is pre-incubated in blocking buffer for 1 hour at room temperature. The blocking buffer consists of 5% nonfat dry milk in TBST. Then the membrane is incubated with anti-human cyclin D1 rabbit polyclonal antibody (Neomarker) and diluted 1:1000 20 overnight at 4° C.

After incubation, the membrane is washed with TBST and subjected to anti-rabbit immunoglobulin G, horseradish peroxidase-linked whole antibody (Amersham Biosciences) diluted 1:2000 as a second antibody for 1 hour at room temperature. Protein-antibody complexes are visualized with an enhanced chemiluminescence Western blot detection and analysis system (Amersham Biosciences Corp).

Alternatively, agents that inhibit EGLN2 expression or activity can be identified using the assays described below. The assays can employ a non-peptide substrate, fully or partially purified polypeptide substrates (purified from cells that naturally express them or produced using recombinant methodologies), cells expressing a polypeptide substrate or and/or cell extracts containing a polypeptide substrate. The assays can be used both to identify agents that decrease hydroxylation of an EGLN2 substrate and agents that increase hydroxylation of an EGLN2 substrate. The substrate for the assay can be a human HIF-1α, a natural substrate of EGLN2 hydroxylation, a surrogate EGLN2 substrate or a fragment thereof that is subject to hydroxylation by EGLN2, for example, a human HIF-1α fragment.

EGLN2 is expected to catalyze the following reaction, in which R is, for example, HIF-1α and ROH is hydroxylated HIF-1α.

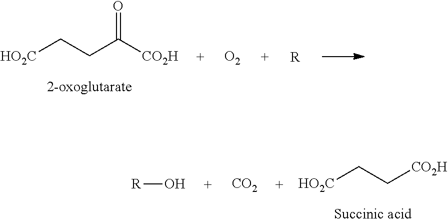

In the assay methods described herein the prolyl hydroxylase (e.g., EGLN2) and the substrate of the hydroxylase (e.g., HIF-1α) are contacted in the presence of a co-substrate, such as 2-oxoglutarate (2OG). The hydroxylase expression or activity can be determined, for example, by determining the turnover of the co-substrate. This may be achieved by determining the presence and/or amount of reaction products, such as hydroxylated substrate or succinic acid. The amount of product may be determined relative to the amount of substrate. Thus, hydroxylase expression or activity may be determined by determining the turnover of 2OG to succinate and CO₂ as described in Myllyharju et al. (EMBO J. 16:1173-1180 (1991)) or as in Cunliffe et al. (Biochem. J. 240:617-619 (1986)), or other suitable assays for CO₂, bicarbonate or succinate production. Such assays can be modified to high throughput format and the invention encompasses such high throughput assays for hydroxylase activity or expression.

An agent which modulates the interaction of HIF-1α or some other substrate of EGLN2 with EGLN2 can be identified by a method comprising: (a) contacting EGLN2 and a test agent in the presence of substrate, e.g., HIF-1α or a fragment thereof, under conditions in which EGLN2 acts on the substrate (e.g., full-length HIF-1α or a fragment thereof that is subject to hydroxylation) in the absence of the test agent; and (b) determining the interaction, or lack of interaction, of EGLN2 and the substrate. The interaction of the hydroxylase with the substrate may be determined by measuring the hydroxylation of the substrate (e.g., using a specific antibody or mass spectroscopy) or the binding of the hydroxylase to the substrate or the level of the substrate in a cell. For example, hydroxylation can decrease the level of the substrate, e.g., HIF-1α in the cell. The interaction can also be measured by measuring any activity related to the action of the hydroxylase on the substrate, such as the levels of co-factors or by-products used or produced in the hydroxylation reaction, or downstream effects mediated through hydroxylation of the substrate.

The assay can be on conversion of the substrate into a detectable product. For example, reverse phase HPLC may be used to separate starting synthetic peptide substrates from the hydroxylated products. Thus, the assay can employ mass spectrometric, spectroscopic, and/or fluorescence techniques as are well known in the art (Masimirembwa et al. (2001) Combinatorial Chemistry & High Throughput Screening 4:245-263, Owicki (2000) J. Biomol. Screen. 5:297-305, Gerslikovich et al. (1996) J. Biochem. Biophys. Meth. 33:135-162, Kraaft et al. (1994) Meth. Enzymol. 241:70-86). The substrate polypeptide, e.g., HIF-1α or a fragment thereof that is hydroxylated by EGLN2, may be immobilized, e.g., on a bead or plate, and hydroxylation of the appropriate residue detected using an antibody or other binding molecule which binds to the hydroxylated polypeptide with a different affinity than to the non-hydroxylated polypeptide. For example, the antibody recognizes hydroxylated HIF-1α, but binds poorly, if at all, to non-hydroxylated HIF-1α. Antibodies that recognize hydroxylated HIF-1α are commercially available and have been described in the art. (See, e.g., Chan et al. (2005) Mol Cell Biol. 25:6415-26.)

Modulators of HIF-1α hydroxylation can also be identified more indirectly by assessing the effect of a test agent on the stability of HIF-1α or the level of HIF-1α activity. Thus, assays can be based on identifying an inhibitor of HIF-1α destruction. Such assays include: (a) providing a HIF-1α substrate that includes a hydroxylation site and providing a hydroxylase under conditions suitable for the hydroxylation of a proline residue in the HIF-1α substrate; (b) providing a test agent, e.g., putative modulator of hydroxylation; and (c) determining whether the HIF-1α has been hydroxylated by assessing HIF-1α activity.

A HIF-1α stabilization assay can be carried out using cells expressing HIF-1α as follows. Cells expressing HIF-1α are seeded into 35 mm culture dishes and grown at 37° C., 20% O₂, 5% CO₂ in standard culture medium, e.g., DMEM, 10% FBS. When cell layers reach confluence, the media is replaced with OPTI-MEM media (Invitrogen Life Technologies, Carlsbad Calif.) and cell layers are incubated for approximately 24 hours at 37° C., 20% O₂, 5% CO₂. A test agent in DMSO or 0.013% DMSO is added to existing medium, and incubation is continued overnight.

Following overnight incubation, the media is removed and the cells are washed two times in cold phosphate buffered saline (PBS) and then lysed in 1 ml of 10 mM Tris (pH 7.4), 1 mM EDTA, 150 mM NaCl, 0.5% IGEPAL (Sigma-Aldrich, St. Louis Mo.), and a protease inhibitor mix (Roche Molecular Biochemicals) for 15 minutes on ice. Cell lysates are centrifuged at 3,000×g for 5 minutes at 4° C., and the cytosolic fractions (supernatant) are collected. The nuclei (pellet) is resuspended and lysed in 100 ml of 20 mM HEPES (pH 7.2), 400 mM NaCl, 1 mM EDTA, 1 mM dithiothreitol, and a protease mix (Roche Molecular Biochemicals), centrifuged at 13,000×g for 5 minutes at 4° C., and the nuclear protein fractions (supernatant) are collected and analyzed for HIF-1α using a QUANTIKINE immunoassay (R&D Systems, Inc., Minneapolis Minn.) according to the manufacturer's instructions

Assays which entail measuring the hydroxylation of a HIF-1α substrate are carried out under conditions in which the hydroxylase can catalyze hydroxylation. Suitable conditions may include pH 6.6 to 8.5 in an appropriate buffer (for example, Tris HCl or MOPS) in the presence of 2-oxoglutarate, dioxygen and preferably ascorbate and ferrous iron. Reducing agents such as dithiothreitol or tris(carboxyethyl)phosphine may also be present to optimize activity. Other enzymes such as catalase and protein disulphide isomerase may be used for the optimization of activity. The enzymes, such as protein disulphide isomerase, may be added in purified or unpurified form. Further components capable of promoting or facilitating the activity of protein disulphide isomerase may also be added.

The format of any of the screening or assay methods may be varied by those of skill in the art. The assays may involve monitoring for hydroxylation of a suitable substrate (in particular monitoring for prolyl hydroxylation), monitoring for the utilization of substrates and co-substrates, monitoring for the production of the expected products between the enzyme and its substrate. Assay methods may also involve screening for the direct interaction between components in the system. Alternatively, assays may be carried out which monitor for downstream effects such as subsequent destruction of HIF-1α, alterations to the levels of HIF-1α in the system and downstream effects mediated by HIF-1α such as HIF-1α mediated transcription using suitable reporter constructs or by monitoring for the upregulation of genes or alterations in the expression patterns of genes know to be regulated directly or indirectly by HIF-1α.

The substrate, enzyme and potential inhibitor agent may be incubated together under conditions which in the absence of inhibitor provide for hydroxylation of a proline within a polypeptide substrate and the effect of the inhibitor may be determined by determining hydroxylation of the substrate. This may be accomplished by any suitable means. Small polypeptide substrates may be recovered and subject to physical analysis, such as mass spectrometry or chromatography, or to functional analysis, such as the ability to bind to VHL (or displace a reporter molecule from VHL) and be targeted for destruction.

The binding of a substrate to a hydroxylase, e.g., EGLN2, can be assessed in vitro by labeling one component with a detectable label and bringing it into contact with the other component which has been immobilized on a solid support. Suitable detectable labels include ³⁵S which may be incorporated into recombinantly produced peptides and polypeptides. Recombinantly produced peptides and polypeptides may also be expressed as a fusion protein containing an epitope which can be labeled with an antibody. Fusion proteins can incorporate six histidine residues at either the N-terminus or C-terminus of the recombinant protein. Such a histidine tag may be used for purification of the protein by using commercially available columns which contain a metal ion, either nickel or cobalt. These tags also serve for detecting the protein using commercially available monoclonal antibodies directed against the six histidine residues. The protein which is immobilized on a solid support may be immobilized using an antibody against that protein bound to a solid support or the protein can be immobilized using other standard methods. A preferred in vitro interaction may utilize a fusion protein including glutathione-S-transferase (GST). This may be immobilized on glutathione agarose beads. In an in vitro assay format of the type described above, a test agent can be assayed by determining its ability to diminish the amount of labeled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined for example, by counting the amount of label present.

The assay can be performed in vivo. The in vivo assay may be performed in a cell line such as a yeast strain in which the relevant polypeptides or peptides are expressed from one or more vectors introduced into the cell.

### Formulations and Modes of Administration

The agents of the present invention can be delivered directly or in pharmaceutical compositions containing excipients, as is well known in the art. The present methods involve administration of an effective amount of an agent of the present invention to a subject.

An effective amount, e.g., dose, of agent or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences; and Hardman, Limbird, and Gilman, eds. (2001) The Pharmacological Basis of Therapeutics.)

For compositions useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a compound, agent, or drug of the present invention refers to an amount or dose of the compound, agent, or drug that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the agent, compound, or pharmaceutical composition that will elicit the biological or medical response of a tissue, system, animal, or human that is being sought by the researcher, veterinarian, medical doctor, or other clinician.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration (MEC). The MEC will vary for each agent or compound but can be estimated from, for example, in vitro data and animal experiments.

Dosages necessary to achieve the MEC will depend on individual characteristics and route of administration. In cases of local administration or selective uptake, the effective local concentration of the drug may not be related to plasma concentration.

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred. For example, for instances in which the agent or compound is not orally bioavailable, intravenous injection may be a preferred route of administration. In certain preferred embodiments, the agents of the present invention are administered orally. In other preferred embodiments, the agents of the present invention are administered by intravenous injection.

Pharmaceutical dosage forms of an agent of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of an agent or a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the agents or compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The agents or compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of an agent or a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

The amount of agent or composition administered may be dependent on a variety of factors, including the sex, age, and weight of the subject being treated, the severity of the affliction, the manner of administration, and the judgment of the prescribing physician.

Inhibitors of EGLN2 enzyme activity or expression can be used alone or in combination with other compounds used to treat various disorders, e.g., cancer. Combination therapies are useful in a variety of situations, including where an effective dose of one or more of the agents used in the combination therapy is associated with undesirable toxicity or side effects when not used in combination. This is because a combination therapy can be used to reduce the required dosage or duration of administration of the individual agents.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising an agent of the invention formulated in a compatible pharmaceutical carrier may also be prepared, placed in an appropriate container, and labeled for treatment of an indicated condition.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### EGLN2 Inhibition Decreases Cyclin D1 Levels in Osteosarcoma Cells

To examine the effect of EGLN2 inhibition on cyclin D1 levels in cells, the following studies were performed. Cultured human osteosarcoma cells (U2OS; ATCC number HTB-96) were incubated with various concentrations of either dimethyl-oxalylglycine (DMOG, 0.2, 0.5, 1.0, or 2.0 mM) or desferrioxamine (DFO, 0.5 mM) for 24 hours. DMOG and DFO inhibit the activity of EGLN enzymes, and inhibition of the activity of EGLN enzymes stabilizes HIF-1α levels in cells. Changes in cyclin D1, HIF-1α, and vinculin (a non-responding control protein) protein levels in the cells were examined by Western blot, using polyclonal antibodies against human cyclin D1 (Lab Vision), HIF-1α (Bethyl Laboratories), and vinculin (Sigma-Aldrich). Western blot analyses were performed using methods previously described by Harlow et al. ((1999) Using antibodies: A laboratory manual. Cold Spring Harbor, N.Y.: Cold Spring Harbor Laboratory Press).

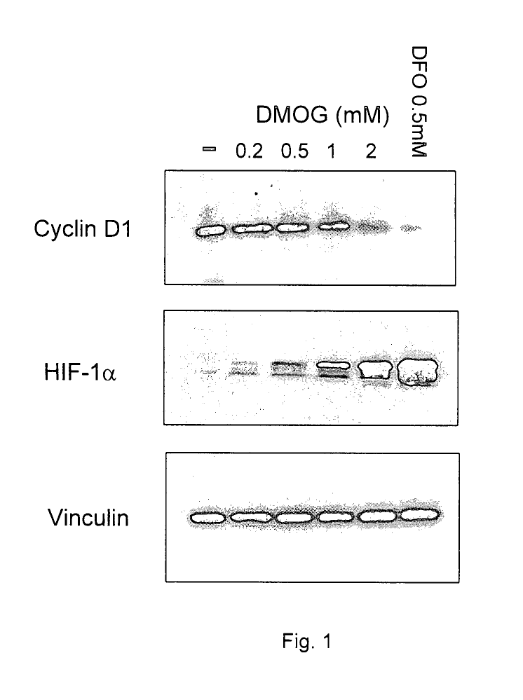

As shown in FIG. 1, U2OS cells incubated with either DFO or various concentrations of DMOG had reduced cyclin D1 protein levels compared to cyclin D1 protein levels observed in non-treated cells. Addition of DMOG to cultured U2OS cells decreased cyclin D1 protein levels in a dose-dependent manner. Additionally, U2OS cells incubated with either DMOG or DFO showed increased HIF-1α protein levels compared to that observed in non-treated cells. (See FIG. 1.) Protein levels for vinculin, a non-responding control protein, were unaltered by either DMOG or DFO addition.

These results showed that DMOG and DFO decreased cyclin D1 protein levels in cultured U2OS cells, a human osteosarcoma cell line. These results indicated that methods of the present invention are useful for reducing cyclin D1 protein levels in cells. As stabilization of HIF-1α by DMOG or DFO is indicative of inhibition of EGLN enzyme activity, these results indicated that inhibition of EGLN2 enzyme activity decreased cyclin D1 protein levels. Taken together, these results showed that methods and agents of the present invention are useful for decreasing cyclin D1 levels by inhibiting EGLN2.

In another series of experiments, the effect of EGLN2 inhibition on cyclin D1 levels in cultured human breast carcinoma cells was examined. Cultured human breast carcinoma cells (ZR-75-1; ATCC number CRL 1500) were incubated with various concentrations of cobalt chloride (CoCl₂,200 μM), dimethyl-oxalylglycine (DMOG, 1 mM), desferrioxamine (DFO, 200 μM), or Compound A (40 μM). All four compounds inhibit the activity of EGLN enzymes, and inhibition of the activity of EGLN enzymes stabilizes HIF-1α levels in cells. Changes in cyclin D1, HIF-1α, and vinculin (a non-responding control protein) protein levels in the cells were examined by Western blot, using polyclonal antibodies against human cyclin D1 (Lab Vision), HIF-1α (Bethyl Laboratories), and vinculin (Sigma-Aldrich). Western blot analyses were performed using methods previously described by Harlow et al. ((1999) Using antibodies: A laboratory manual. Cold Spring Harbor, N.Y.: Cold Spring Harbor Laboratory Press).

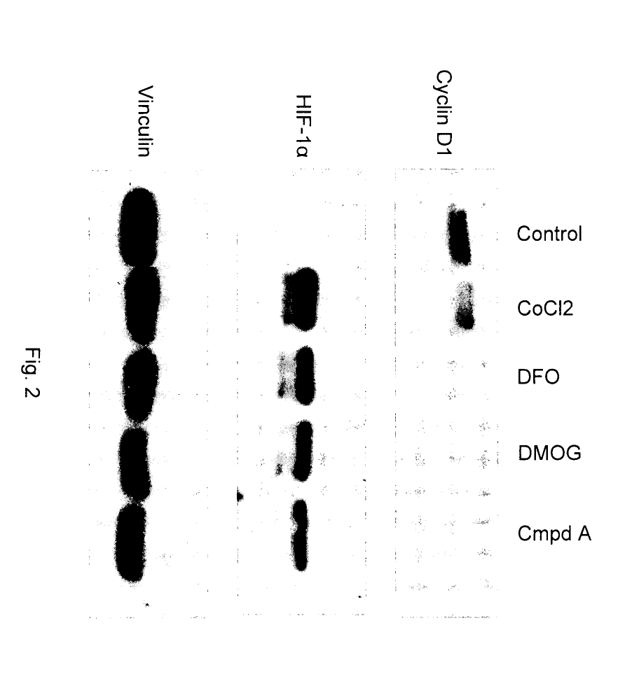

As shown in FIG. 2, human breast carcinoma cells incubated with CoCl₂, DFO, DMOG, or Compound A had reduced cyclin D1 protein levels compared to cyclin D1 protein levels observed in non-treated control cells. Additionally, human breast carcinoma cells incubated with CoCl₂, DFO, DMOG, or Compound A showed increased HIF-1α protein levels compared to that observed in non-treated cells. (See FIG. 2.) Protein levels for vinculin, a non-responding control protein, were unaltered by CoCl₂, DFO, DMOG, or Compound A addition.

These results showed that CoCl₂, DFO, DMOG, and Compound A (inhibitors of the activity of EGLN enzymes) decreased cyclin D1 protein levels in cultured ZR-75-1 cells, a human breast carcinoma cell line. These results indicated that methods of the present invention are useful for reducing cyclin D1 protein levels in cells. As stabilization of HIF-1α by CoCl₂, DFO, DMOG, or Compound A is indicative of inhibition of EGLN enzyme activity, these results indicated that inhibition of EGLN2 enzyme activity decreased cyclin D1 protein levels. Taken together, these results showed that methods and agents of the present invention are useful for decreasing cyclin D1 levels by inhibiting EGLN2.

### Example 2

### EGLN2 siRNA Decreases Cyclin D1 Levels in U2OS Cells and HeLa Cells

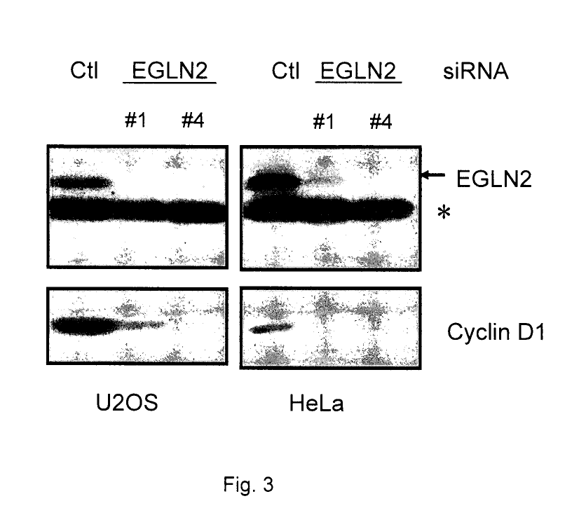

To examine the effect of inhibiting EGLN2 expression and activity on cyclin D1 protein levels, the following studies were performed. Human osteosarcoma cells (U2OS) and human cervical adenocarcinoma cells (HeLa; ATCC number CCL-2) were transfected with one of two different siRNA expression constructs directed against EGLN2 mRNA (designated #1 and #4 in FIG. 3) or with control siRNA (Ctl in FIG. 3) using oligofectamine (Invitrogen). The siRNA constructs used in these studies were as follows:

| EglN2 #1: | |
|---|---|
| 5′-GACTATATCGTGCCCTGCATG-3′ | (SEQ ID NO: 1) |
| EglN2 #4: | |
| 5′-GCCACTCTTTGACCGGTTGCT-3′ | (SEQ ID NO: 2) |
| Ctl: | |
| 5′-AACAGTCGCGTTTGCGACTGG-3′ | (SEQ ID NO: 3) |

Cyclin D1 and EGLN2 protein levels were examined by Western blot analysis using polyclonal antibodies against human cyclin D1 (Lab Vision) and EGLN2 (Novus Biologicals). Western blot analyses were performed as described above in Example 1.

As shown in FIG. 3, U2OS cells and HeLa cells transfected with siRNA directed against EGLN2 mRNA had reduced EGLN2 protein levels compared to EGLN2 protein levels observed in these cells transfected with control siRNA. These results indicated that the siRNAs directed against EGLN2 mRNA were effective at reducing EGLN2 expression. U2OS cells and HeLa cells transfected with siRNA directed against EGLN2 mRNA had reduced cyclin D1 protein levels compared to cyclin D1 protein levels observed in these cells transfected with control siRNA. A nonspecific protein band (designated * in FIG. 3), used here as a negative loading control, was unchanged by transfection of the cells with either EGLN2 siRNA or control siRNA.

These results showed that siRNA directed against EGLN2 mRNA reduced cyclin D1 protein levels in cultured human osteosarcoma cells (U2OS) and human cervical adenocarcinoma cells (HeLa). These results indicated that inhibition of EGLN2 expression decreased cyclin D1 protein levels. Taken together, these results showed that methods and agents of the present invention are useful for decreasing cyclin D1 levels by inhibiting EGLN2.

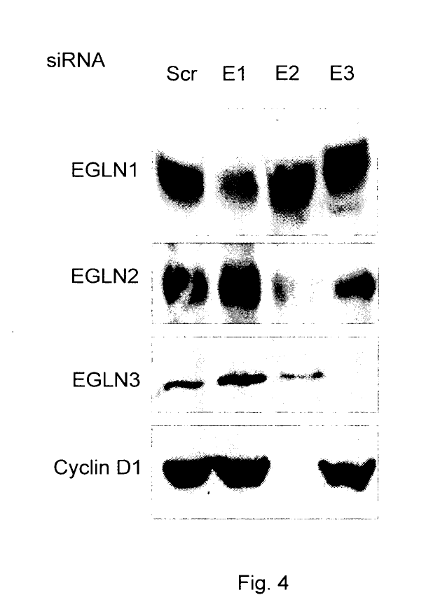

In another series of experiments, the effect of EGLN1, EGLN2, and EGLN3 reduction on cyclin D1 levels in a human breast carcinoma cell line was determined. Human cervical adenocarcinoma cells (HeLa; ATCC number CCL-2) were transfected with one of four different siRNA expression constructs directed against EGLN1 mRNA (designated E1 in FIG. 4), EGLN2 mRNA (E2 in FIG. 4), EGLN3 mRNA (E3 in FIG. 4) or with control siRNA (Scr in FIG. 4) using oligofectamine (Invitrogen). The siRNA constructs used in these studies were as follows:

| E1: | |
|---|---|
| 5′-AGCTCCTTCTACTGCTGCA-3′ | (SEQ ID NO: 12) |
| E2: | |
| 5′-GCCACTCTTTGACCGGTTGCT-3′ | (SEQ ID NO: 2) |
| E3: | |
| 5′-CAGGTTATGTTCGCCACGT-3′ | (SEQ ID NO: 13) |
| Scr: | |
| 5′-AACAGTCGCGTTTGCGACTGG-3′ | (SEQ ID NO: 3) |

Cyclin D1, EGLN1, EGLN2, and EGLN3 protein levels were examined by Western blot analysis using polyclonal antibodies against human EGLN1, EGLN2, EGLN3 (Novus Biologicals), and cyclin D1 (Lab Vision). Western blot analyses were performed as described above in Example 1.

As shown in FIG. 4, HeLa cells transfected with siRNA directed against EGLN1, EGLN2, or EGLN3 mRNA had reduced EGLN1, EGLN2, and EGLN3 protein levels, respectively, compared to the corresponding EGLN protein levels observed in HeLa cells transfected with control siRNA. These results indicated that the siRNAs directed against EGLN1, EGLN2, and EGLN3 mRNA were effective at reducing EGLN1, EGLN2, and EGLN3 expression, respectively. Only the HeLa cells transfected with siRNA directed against EGLN2 mRNA had reduced cyclin D1 protein levels compared to cyclin D1 protein levels observed in HeLA cells transfected with EGLN1, EGLN3, or control siRNA (see FIG. 4).

These results showed that siRNA directed against EGLN2 mRNA, but not EGLN1 or EGLN3 mRNA, reduced cyclin D1 protein levels in cultured human cervical adenocarcinoma cells (HeLa). These results indicated that inhibition of EGLN2 expression decreased cyclin D1 protein levels. Taken together, these results showed that methods and agents of the present invention are useful for decreasing cyclin D1 levels by inhibiting EGLN2.

### Example 3

### EGLN2 siRNA Decreases Cyclin D1 Expression in U2OS Cells and HeLa Cells

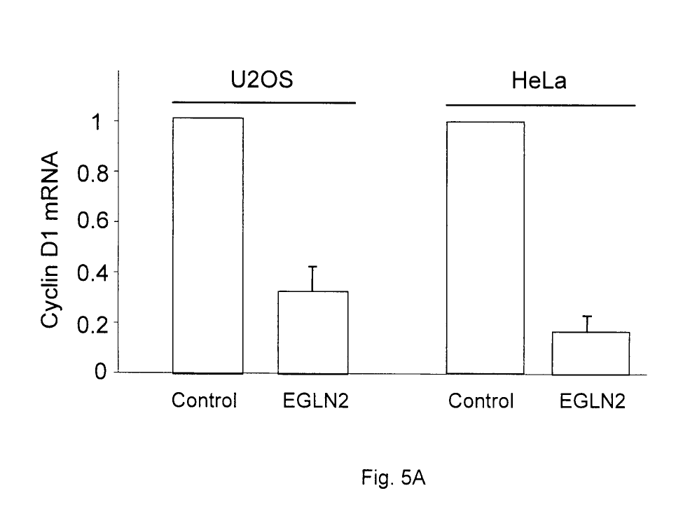

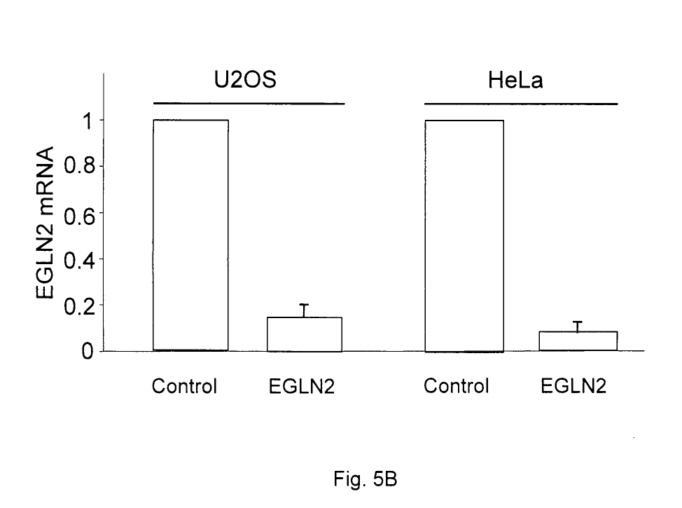

To examine the effect of inhibiting EGLN2 expression and activity on cyclin D1 expression, the following studies were performed. Human osteosarcoma cells (U2OS) and human cervical adenocarcinoma cells (HeLa; ATCC number CCL-2) were transfected with an siRNA expression construct directed against EGLN2 mRNA (designated EGLN2 in FIGS. 5A and 5B) or with control siRNA (Control in FIGS. 5A and 5B) using oligofectamine (Invitrogen). The siRNA constructs used in these studies were as follows:

| EGLN2: | |
|---|---|
| 5′-GCCACTCTTTGACCGGTTGCT-3′ | (SEQ ID NO: 2) |
| Control: | |
| 5′-AACAGTCGCGTTTGCGACTGG-3′ | (SEQ ID NO: 3) |

Following transfection with siRNA, U2OS and HeLa cells were lysed in RIPA buffer and mRNA was extracted using an RNAeasy Kit (Qiagen) according to the manufacture's instructions. EGLN2 and cyclin D1 mRNA levels were measured quantitatively using RT-PCR. Briefly, samples were heated to 95° C. for 15 minutes and then cycled through 95° C. for 30 seconds, 55° C. for 30 seconds, and 72° C. for 30 seconds for a total of 40 cycles. Primers used in these studies were as follows:

Cyclin D1:
(SEQ ID NO: 4)
5′-AAACAGATCATCCGCAAACACGTGTGAGGCGGTAGTAGGACA-3′
hEGLN2:
(SEQ ID NO: 5)
5′-AACATCGAGCCACTCTTTGAC-3′
hEGLN2:
(SEQ ID NO: 6)
5′-TCCTTGGCATCAAAATACCAG-3′
h18S rRNA-forward:
(SEQ ID NO: 7)
5′-AAGACGATCAGATACCGTCGTAG-3′
h18S rRNA-reverse:
(SEQ ID NO: 8)
5′-GTTTCAGCTTTGCAACCATACTC-3′

As shown in FIG. 5B, U2OS cells and HeLa cells transfected with siRNA directed against EGLN2 mRNA had reduced EGLN2 mRNA levels compared to EGLN2 mRNA levels observed in cells transfected with control siRNA. These results showed that siRNA directed against EGLN2 mRNA reduced EGLN2 mRNA levels in cultured human osteosarcoma cells (U2OS) and human cervical adenocarcinoma cells (HeLa). FIG. 5A shows that U2OS cells and HeLa cells transfected with siRNA directed against EGLN2 mRNA had reduced cyclin D1 mRNA levels compared to cyclin D1 mRNA levels observed in cells transfected with control siRNA. Data for mRNA levels for EGLN2 and cyclin D1 (shown in FIGS. 5A and 5B) were normalized to that of 18S ribosomal RNA within each sample.

These results showed that inhibition or reduction of EGLN2 mRNA levels (by transfection of siRNA directed against EGLN2 mRNA) reduced cyclin D1 mRNA levels in cultured human osteosarcoma cells (U2OS) and human cervical adenocarcinoma cells (HeLa). These results indicated that inhibition of EGLN2 expression decreased cyclin D1 mRNA levels. Taken together, these results showed that methods and agents of the present invention are useful for decreasing cyclin D1 expression by inhibiting EGLN2.

### Example 4

### EGLN2 Increases Cyclin D1 Promoter Activity in U2OS Cells

To examine the effect of EGLN2 expression and activity on cyclin D1 expression, the following studies were performed. Human osteosarcoma cells (U2OS) were co-transfected with the following: an expression vector directing the expression of firefly luciferase under the control of the cyclin D1 promoter; an expression vector directing the expression of renilla luciferase (a control used for normalization purposes); and various amounts of either an expression vector directing the expression of FLAG-EGLN2 (0, 0.02, 0.01, 0.5 μg; SEQ ID NO:9) or an expression vector directing the expression of a mutant EGLN2 (where a conserved histidine residue within the EGLN2 catalytic domain has been changed to alanine, H358A). Cyclin D1 promoter activity was measured using a luciferase dual reporter assay (Promega) according to the manufacture's instructions.

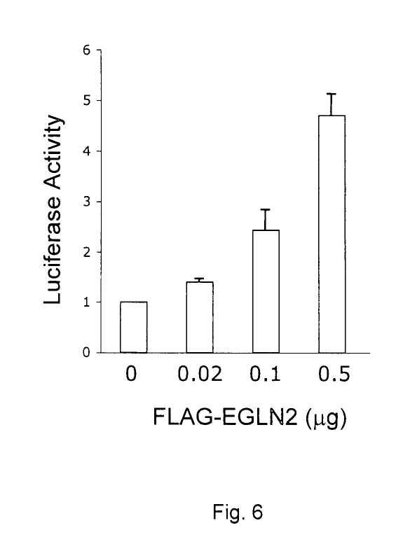

As shown in FIG. 6, U2OS cells transfected with a FLAG-EGLN2 expression vector showed increased cyclin D1 promoter activity compared to the cyclin D1 promoter activity observed in cells that were not transfected with FLAG-EGLN2 expression vector. Transfection of U2OS cells with FLAG-EGLN2 increased cyclin D1 promoter activity in a dose-dependent manner, as cells transfected with increasing amounts of FLAG-EGLN2 vector showed increased cyclin D1 promoter activity. U2OS cells transfected with an EGLN2 mutant expression vector (i.e., H358A) showed lower cyclin D1 promoter activity compared to that observed in cells transfected with an equivalent amount of FLAG-EGLN2 vector. (Data not shown.)

These results showed that a FLAG-EGLN2 vector (directing increased expression of EGLN2) increased expression of cyclin D1 in a dose dependent manner. These results also showed that increased EGLN2 expression resulted in increased cyclin D1 expression, suggesting that increased EGLN2 activity increased cyclin D1 levels in cells. These results provided further support that cyclin D1 expression and activity can be regulated by modulation of EGLN2 expression and activity.

### Example 5

### Inhibition of Estrogen-Induced Expression of Cyclin D1 in Breast Carcinoma Cells

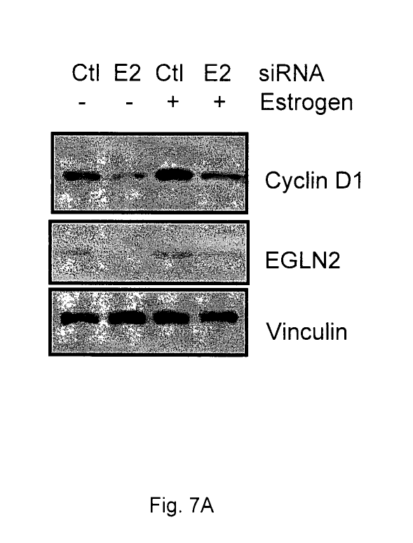

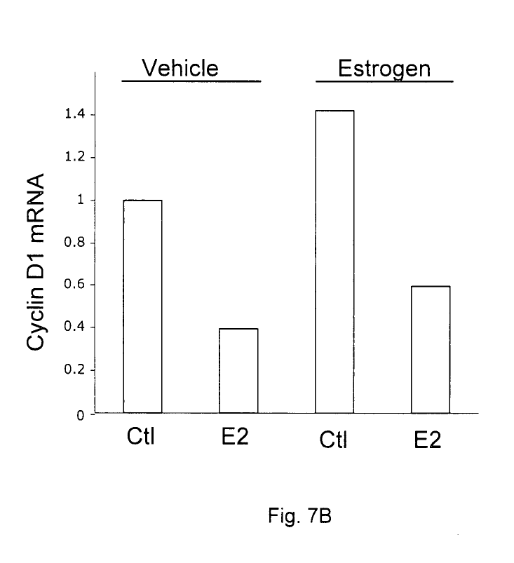

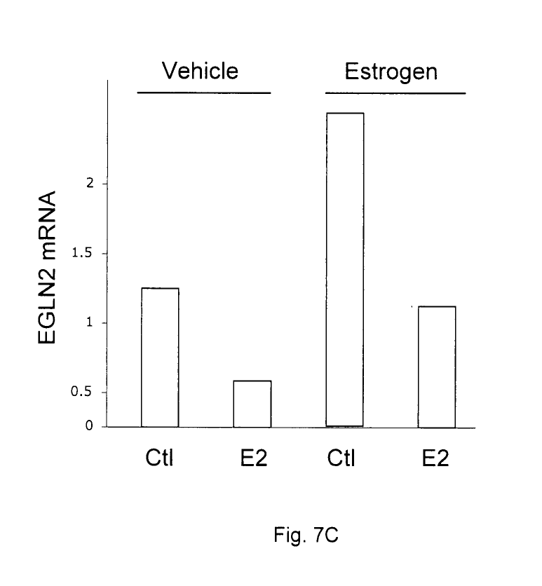

To examine the effect of EGLN2 expression and activity on estrogen-induced increases in cyclin D1 expression, the following experiments were performed. Estrogen increases cyclin D1 levels in breast cancer cells (T47D) in vitro. Human breast carcinoma cells (T47D; ATCC number HTB-133) used in these studies were transfected with an siRNA expression construct directed against EGLN2 mRNA (designated E2 in FIGS. 7A, 7B, and 7C) or with control siRNA (Ctl in FIGS. 7A, 7B, and 7C). The siRNA constructs used in these studies were as follows:

| EGLN2: | |
|---|---|
| 5′-GCCACTCTTTGACCGGTTGCT-3′ | (SEQ ID NO: 2) |
| Control: | |
| 5′-AACAGTCGCGTTTGCGACTGG-3′ | (SEQ ID NO: 3) |

Forty-eight hours after transfection, cells were incubated with estrogen (10 nM in 3 mL medium) or a vehicle (3 mL medium) for 8 hours. The cells were then lysed in RIPA buffer. Protein was extracted from the lysate using EBC buffer (50 mM Tris-HCl pH 8.0; 120 mM NaCl; 0.5% NP40; 1× protease inhibitors); mRNA was extracted from the lysate using an RNAeasy kit (Qiagen) according to the manufacture's instructions. Following extraction, mRNA and protein expression levels were determined using RT-PCR and Western blot analysis, respectively. RT-PCR was carried out as described above in Example 3. Western bolt analyses were performed as described above in Example 2 using polyclonal antibodies against human cyclin D1 (Lab Vision), EGLN2 (Novus Biologicals), and vinculin (Sigma-Aldrich).

As shown in FIG. 7A, addition of estrogen to T47D cells transfected with control siRNA increased EGLN2 and cyclin D1 protein levels compared that observed in vehicle-treated (non-estrogen) T47D cells transfected with control siRNA. T47D cells transfected with EGLN2 siRNA (E2 in FIG. 7A) did not show an increase in cyclin D1 protein levels upon estrogen addition. (See FIG. 7A.) These results indicated that inhibition of EGLN2 expression by transfection with EGLN2 siRNA inhibited the estrogen-induced increase in cyclin D1 levels. Vinculin protein levels, used here as a negative control, were unchanged by estrogen treatment.

As shown in FIGS. 7B and 7C, addition of estrogen to T47D cells transfected with control siRNA increased EGLN2 mRNA and cyclin D1 mRNA levels compared that observed in vehicle-treated (non-estrogen) T47D cells transfected with control siRNA. T47D cells transfected with EGLN2 siRNA did not show an increase in cyclin D1 mRNA levels upon estrogen addition. (See FIG. 7B.) These results indicated that inhibition of EGLN2 expression by transfection with EGLN2 siRNA inhibited the estrogen-induced expression of cyclin D1 mRNA.

These results showed that inhibition of EGLN2 was effective at reducing estrogen-induced increases in cyclin D1 mRNA and protein levels in cultured human breast cancer cells (T47D). These results indicated that methods of the present invention are useful for reducing cyclin D1 in cells by inhibiting EGLN2 activity.

Taken together, these results indicated that the present methods and agents are useful for treating disorders, such as cancer, associated with elevated cyclin D1 levels. Additionally, these results further suggested that methods and agents of the present invention are useful for reducing cyclin D1 in estrogen receptor (ER)-positive cells and for treating estrogen receptor (ER)-positive cancers.

In another series of experiments, the effect of EGLN2 reduction on cyclin D1 levels in a human breast carcinoma cell line was determined. Human breast carcinoma cells (ZR-75-1; ATCC number CRL-1500) were infected with one of two retroviral vectors encoding short hairpin RNAs directed against EGLN2 mRNA (designated E2 shRNA(A) and E2 shRNA(4) in FIG. 8) or with control shRNA (Scr in FIG. 8). The shRNA constructs used in these studies were as follows:

| E2 shRNA(A): | |
|---|---|
| 5′-GACTATATCGTGCCCTGCATG-3′ | (SEQ ID NO: 1) |
| E2 shRNA(4): | |
| 5′-GCCACTCTTTGACCGGTTGCT-3′ | (SEQ ID NO: 2) |
| Scr: | |
| 5′-AACAGTCGCGTTTGCGACTGG-3′ | (SEQ ID NO: 3) |

Following infection with shRNA, ZR-75-1 cells were incubated under standard cell culture conditions and proteins were extracted as described above. Cyclin D1 and EGLN2 protein levels were examined by Western blot analysis using polyclonal antibodies against human cyclin D1 (Lab Vision), EGLN2 (Novus Biologicals) and vinculin (Sigma-Aldrich). Western blot analyses were performed as described above in Example 1.

As shown in FIG. 8, ZR-75-1 cells infected with retroviral vectors encoding shRNA directed against EGLN2 mRNA had reduced EGLN2 protein levels compared to EGLN2 protein levels observed in these cells infected with control shRNA. These results indicated that the shRNAs directed against EGLN2 mRNA were effective at reducing EGLN2 expression. ZR-75-1 cells infected with retroviral vectors encoding shRNA directed against EGLN2 mRNA had reduced cyclin D1 protein levels compared to cyclin D1 protein levels observed in these cells infected with control shRNA.

These results showed that shRNA directed against EGLN2 mRNA reduced cyclin D1 protein levels in cultured human breast carcinoma cells (ZR-75-1). These results further showed that methods and agents of the present invention are effective at reducing cyclin D1 levels in breast cancer cells. Additionally, these results suggested that methods and agents of the present invention are useful for reducing cyclin D1 in estrogen receptor (ER)-positive cells and for treating estrogen receptor (ER)-positive cancers.

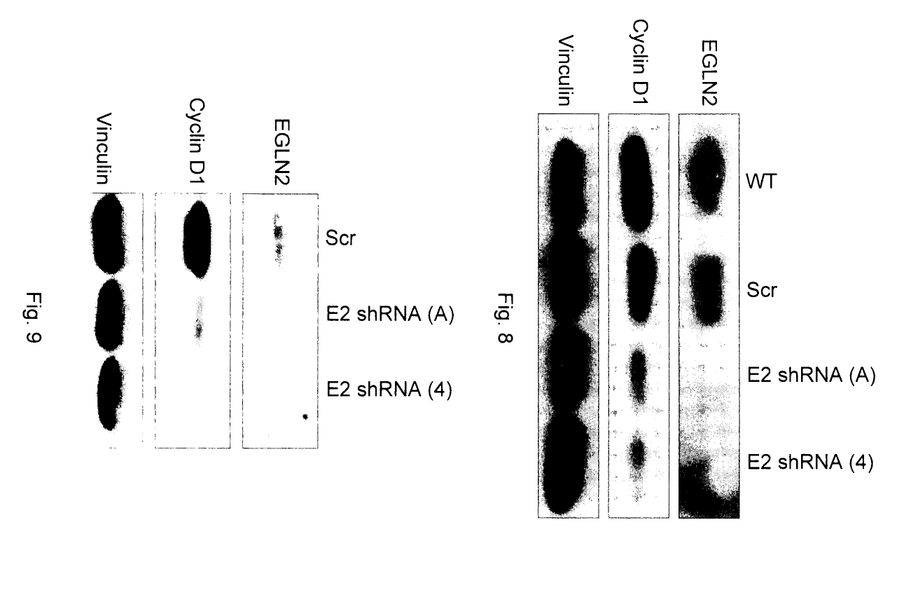

In another series of experiments, the effect of EGLN2 reduction on cyclin D1 levels in an additional human breast carcinoma cell line was determined. Human breast carcinoma cells (BT-474; ATCC number HTB-20) were infected with one of two retroviral vectors encoding short hairpin RNAs directed against EGLN2 mRNA (designated E2 shRNA(A) and E2 shRNA(4) in FIG. 9) or with control shRNA (Scr in FIG. 9). The shRNA constructs used in these studies were as follows:

| E2 shRNA(A): | |
|---|---|
| 5′-GACTATATCGTGCCCTGCATG-3′ | (SEQ ID NO: 1) |
| E2 shRNA(4): | |
| 5′-GCCACTCTTTGACCGGTTGCT-3′ | (SEQ ID NO: 2) |
| Scr: | |
| 5′-AACAGTCGCGTTTGCGACTGG-3′ | (SEQ ID NO: 3) |

Following infection with shRNA, BT-474 cells were incubated under standard cell culture conditions and proteins were extracted as described above. Cyclin D1, EGLN2, and vinculin protein levels were examined by Western blot analysis using polyclonal antibodies against human cyclin D1 (Lab Vision), EGLN2 (Novus Biologicals) and vinculin (Sigma-Aldrich). Western blot analyses were performed as described above in Example 1.

As shown in FIG. 9, BT-474 cells infected with retroviral vectors encoding shRNA directed against EGLN2 mRNA had reduced EGLN2 protein levels compared to EGLN2 protein levels observed in these cells infected with control shRNA. These results indicated that the shRNAs directed against EGLN2 mRNA were effective at reducing EGLN2 expression. BT-474 cells infected with retroviral vectors encoding shRNA directed against EGLN2 mRNA had reduced cyclin D1 protein levels compared to cyclin D1 protein levels observed in these cells infected with control shRNA (see FIG. 9).

These results showed that shRNA directed against EGLN2 mRNA reduced cyclin D1 protein levels in cultured human breast carcinoma cells (BT-474). These results further showed that methods and agents of the present invention are effective at reducing cyclin D1 levels in breast cancer cells. Additionally, these results suggested that methods and agents of the present invention are useful for reducing cyclin D1 in estrogen receptor (ER)-positive cells and for treating estrogen receptor (ER)-positive cancers.

### Example 6

### EGLN2 shRNA Reduces Proliferation of Breast Carcinoma Cells

To examine the effect of inhibiting EGLN2 expression and activity on cyclin D1 levels involved in cell-cycle progression, the following studies were performed. Ecotropic retroviruses used in these studies for RNA interference (RNAi) were prepared by using a pMKO retroviral vector that contains a gene for puromycin resistance. The RNAi sense strands used in these studies were as follows:

| shEGLN2-A: | |
|---|---|
| 5′-GACTATATCGTGCCCTGCATG-3′ | (SEQ ID NO: 1) |
| shEGLN2-B: | |
| 5′-GCCACTCTTTGACCGGTTGCT-3′ | (SEQ ID NO: 2) |
| shGFP: | |
| 5′-GGCTACGTCCAGGAGCGCACC-3′ | (SEQ ID NO: 10) |

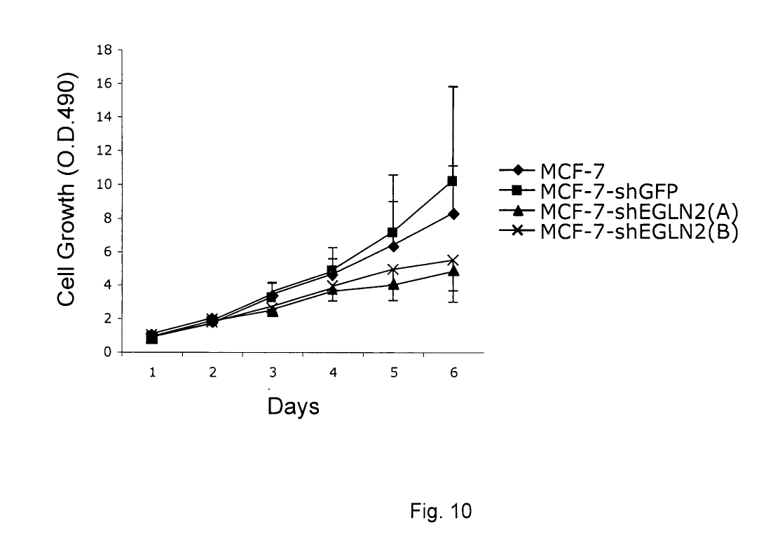

Virus stocks were prepared using methods previously described. (Li et al. (2007) Mol Cell Biol 27:5381-5392.) Briefly, oligonucleotides encoding shRNA targeting EGLN2 or GFP were ligated into the pMKO retroviral vector according to the manufacturer's instructions. Phoenix packaging cells were transfected with the pMKO vectors using Lipofectamine 2000 (Invitrogen). Human breast carcinoma cells (MCF-7; ATCC number HTB-22) were infected three times over three days by incubation with retrovirus containing sequences encoding either one of two different shRNA expression constructs directed against EGLN2 mRNA (shEGLN2(A) (triangles in FIG. 10) and shEGLN2(B) (x's in FIG. 10)); with an shRNA expression construct directed against GFP mRNA (shGFP (squares in FIG. 10)); or with control shRNA (diamonds in FIG. 10). After incubation, infected MCF-7 cells were selected by growth in the presence of puromycin (2 μg/ml). Cell proliferation was measured daily for six days beginning one day after selection with puromycin using a Cell Proliferation Kit II (XTT) (Roche Diagnostics) according to the manufacturer's instructions.

As shown in FIG. 10, cell growth was reduced in MCF-7 cells infected with retrovirus encoding either shRNA directed against EGLN2 mRNA compared to the cell growth observed in MCF-7 cells infected with control shRNA. Growth of cells infected with GFP shRNA, used here as a negative control, was similar to the proliferation observed in cells transfected with control shRNA. (See FIG. 10.)

There results showed that shRNA directed against EGLN2 reduced cell growth and proliferation in human breast carcinoma cells (MCF-7). These results indicated that methods and agents of the present invention are effective at reducing cell growth and proliferation. These results also indicated that methods and of the present invention are effective at reducing cell growth and proliferation by inhibiting EGLN2 activity.

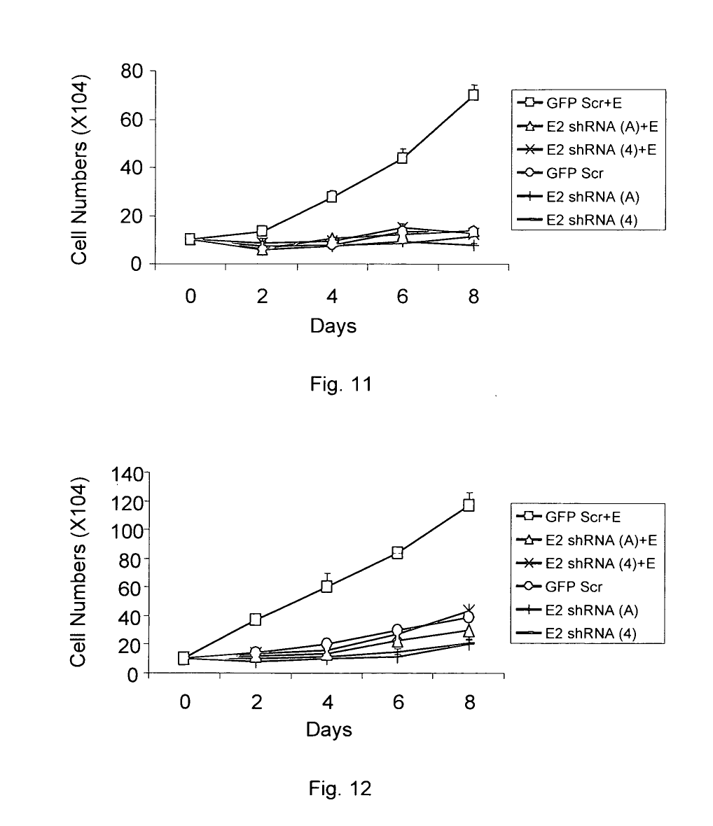

In another series of experiments, the effect of inhibiting EGLN2 expression and activity on proliferation of estrogen-dependent human breast carcinoma cells was examined. Human breast carcinoma cells (ZR-75-1; ATCC number CRL-1500) were infected with one of two retroviral vectors encoding short hairpin RNAs directed against EGLN2 mRNA (designated E2 shRNA(A) and E2 shRNA(4) in FIG. 11) or with control shRNA (GFP Scr in FIG. 11). The shRNA constructs used in these studies were as follows:

| E2 shRNA(A): | |
|---|---|
| 5′-GACTATATCGTGCCCTGCATG-3′ | (SEQ ID NO: 1) |
| E2 shRNA(4): | |
| 5′-GCCACTCTTTGACCGGTTGCT-3′ | (SEQ ID NO: 2) |
| GFP Scr: | |
| 5′-AACAGTCGCGTTTGCGACTGG-3′ | (SEQ ID NO: 3) |

Cells were grown in the presence or absence of estrogen (10 nM) and cell proliferation was measured every other day for eight days using a Cell Proliferation Kit II (XTT) (Roche Diagnostics) according to the manufacturer's instructions.

As shown in FIG. 11, ZR-75-1 cells proliferated in the presence of estrogen but not in its absence. Cell proliferation in the presence of estrogen was reduced, however, in ZR-75-1 cells infected with either of the two EGLN2 shRNAs (see FIG. 11).

There results showed that shRNA directed against EGLN2 reduced cell growth and proliferation in estrogen-dependent human breast carcinoma cells (ZR-75-1). These results indicated that methods and agents of the present invention are effective at reducing cell growth and proliferation. These results also indicated that methods and of the present invention are effective at reducing cancer cell growth and proliferation by inhibiting EGLN2 activity.

In another series of experiments, the effect of inhibiting EGLN2 expression and activity on proliferation of an additional estrogen-dependent human breast carcinoma cell line was examined. Human breast carcinoma cells (BT-474; ATCC number HTB-20) were infected with one of two retroviral vectors encoding short hairpin RNAs directed against EGLN2 mRNA (designated E2 shRNA(A) and E2 shRNA(4) in FIG. 12) or with control shRNA (GFP Scr in FIG. 12). The shRNA constructs used in these studies were as follows:

| E2 shRNA(A): | |
|---|---|
| 5′-GACTATATCGTGCCCTGCATG-3′ | (SEQ ID NO: 1) |
| E2 shRNA(4): | |
| 5′-GCCACTCTTTGACCGGTTGCT-3′ | (SEQ ID NO: 2) |
| GFP Scr: | |
| 5′-AACAGTCGCGTTTGCGACTGG-3′ | (SEQ ID NO: 3) |

Cells were grown in the presence or absence of estrogen (10 nM) and cell proliferation was measured every other day for eight days using a Cell Proliferation Kit II (XTT) (Roche Diagnostics) according to the manufacturer's instructions.

As shown in FIG. 12, BT-474 cells proliferated in the presence of estrogen but not in its absence. Cell proliferation in the presence of estrogen was reduced, however, in BT-474 cells infected with either of the two EGLN2 shRNAs (see FIG. 12).

There results showed that shRNA directed against EGLN2 reduced cell growth and proliferation in estrogen-dependent human breast carcinoma cells (BT-474). These results indicated that methods and agents of the present invention are effective at reducing cell growth and proliferation. These results also indicated that methods and of the present invention are effective at reducing cancer cell growth and proliferation by inhibiting EGLN2 activity.

### Example 7

### Reduced Protein Levels of Cyclin D1 in Breast Carcinoma Cells

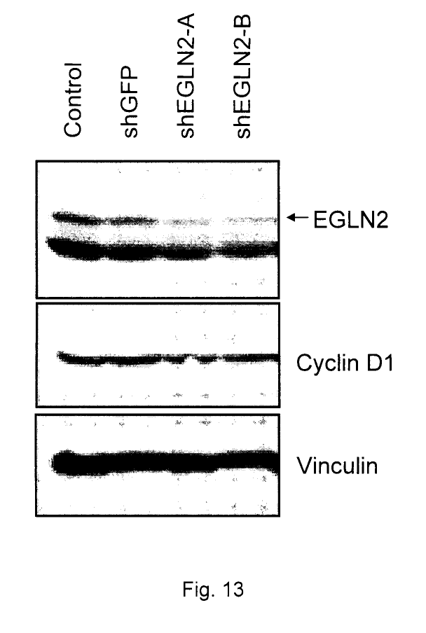

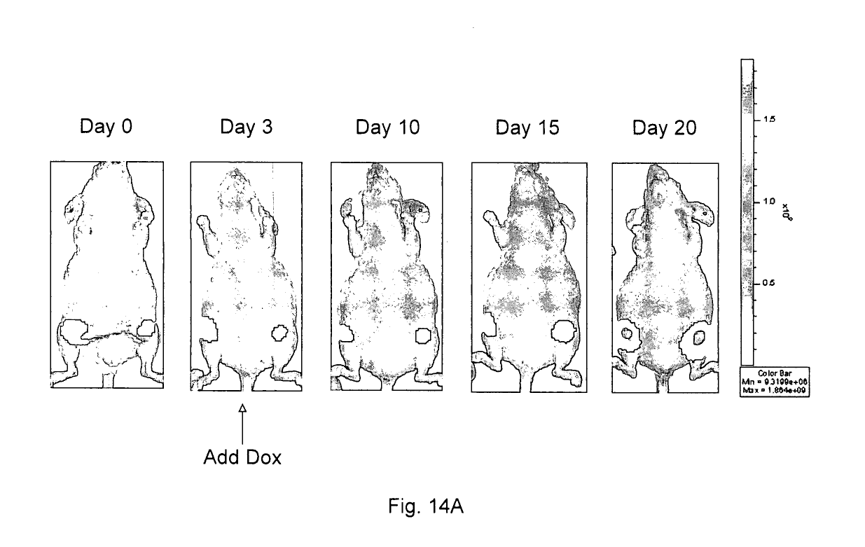

To examine the effect of inhibiting EGLN2 expression and activity on cyclin D1 protein levels, the following studies were performed. Human breast carcinoma cells (MCF-7; ATCC number HTB-22) used in these studies were transfected with one of two different shRNA expression constructs directed against EGLN2 mRNA (designated shEGLN2-A and shEGLN2-B in FIG. 13); an shRNA expression construct directed against GFP mRNA (designated shGFP in FIG. 13); or with an expression construct directed to control shRNA (designated Control in FIG. 13) using oligofectamine (Invitrogen). The RNAi sense strands used in these studies were as follows:

| shEGLN2-A: | |
|---|---|
| 5′-GACTATATCGTGCCCTGCATG-3′ | (SEQ ID NO: 1) |
| shEGLN2-B: | |
| 5′-GCCACTCTTTGACCGGTTGCT-3′ | (SEQ ID NO: 2) |
| shGFP: | |
| 5′-GGCTACGTCCAGGAGCGCACC-3′ | (SEQ ID NO: 10) |

Protein levels for cyclin D1, EGLN2, and vinculin (which served as a non-responding control) were analyzed by Western blot analysis using polyclonal antibodies against human cyclin D1 (Lab Vision), EGLN2 (Novus Biologicals), and vinculin (Sigma-Aldrich). Western blot analyses were performed as described above in Example 1.

As shown in FIG. 13, EGLN2-transfected MCF-7 cells transfected with an shRNA expression construct directed against EGLN2 mRNA showed reduced EGLN2 and cyclin D1 protein levels compared to EGLN2 and cyclin D1 protein levels observed in cells transfected with either control or GFP shRNA expression constructs. Vinculin protein levels, used here as a negative control, were unaltered by transfection with any shRNA.

These results showed that shRNA directed against EGLN2 reduced both EGLN2 and cyclin D1 protein levels in cultured MCF-7 cells, a human breast carcinoma cell line. These results indicated that methods and agents of the present invention are useful for reducing cyclin D1 protein levels in cells, including cancer cells. These results also indicated that methods and agents of the present invention are effective at reducing cyclin D1 by inhibiting EGLN2 activity.

### Example 8

### Reduced EGLN2 Expression Decreased Tumor Formation In Vivo

To examine the effect of inhibiting EGLN2 expression and activity on tumor formation in vivo, the following studies were performed in a xenograft tumor model. Six-week old female swiss nude mice were used in these xenograft studies. The shRNA constructs used in these studies were as follows:

| Egln2 shRNA: | |
|---|---|
| 5′-GCCACTCTTTGACCGGTTGCT-3′ | (SEQ ID NO: 2) |
| Scr: | |
| 5′-AACAGTCGCGTTTGCGACTGG-3′ | (SEQ ID NO: 3) |

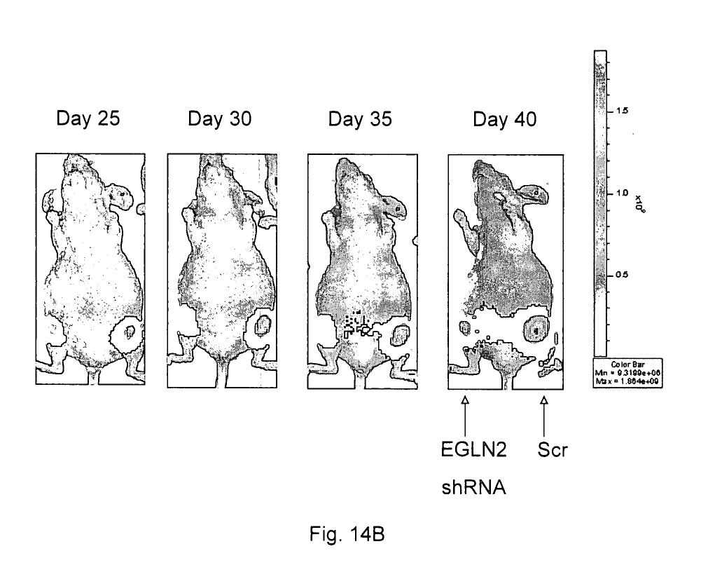

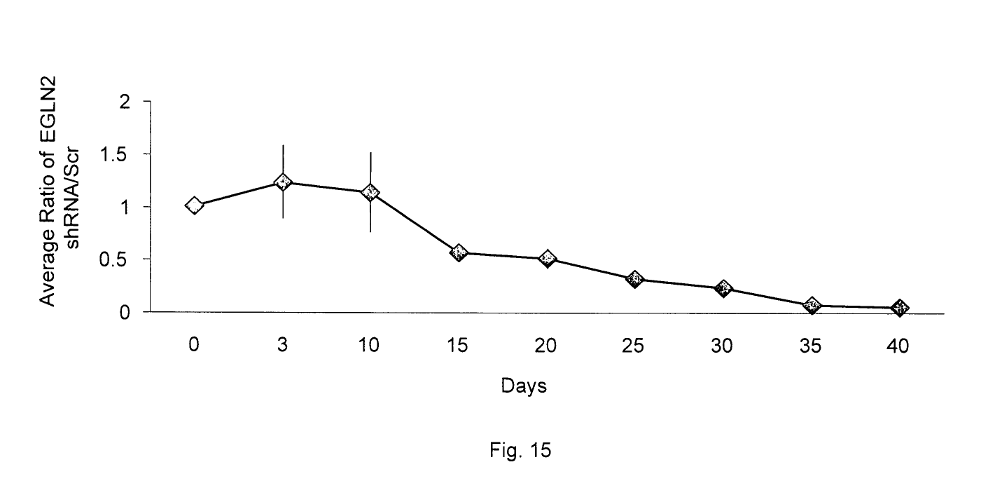

Human breast carcinoma cells (ZR-75-1; ATCC number CRL-1500) previously infected with doxycycline-inducible lentiviruses encoding shRNAs against EGLN2 (Egln2 shRNA) or scrambled control (Scr) were infected with a retrovirus encoding luciferase and injected orthotopically into the 3^{rd} mammary glands of the immunocompromised mice. One mammary gland was injected with ZR-75-1 cells containing the doxycycline-inducible EGLN2 shRNA and the contralateral mammary gland was injected with ZR-75-1 cells containing the doxycycline-inducible control shRNA (Scr). Mice were treated with a depot of estrogen to promote tumor growth and tumor burden was monitored non-invasively by bioluminescent imaging beginning one week after cell implantation (day 0 in FIGS. 14A and 15). Three days later (day 3 in FIGS. 14A and 15) mouse chow was supplements with doxycycline and continued for the duration of the study. Tumor burden was monitored at various days by bioluminescence.

For bioluminescent detection and quantification of cancer cells and relative tumor mass noninvasively, mice were given a single i.p. injection of a mixture of luciferin (50 mg/kg) ketamine (150 mg/kg) and xylazine (12 mg/kg) in sterile water. Fifteen minutes later, mice were placed in a light-tight chamber equipped with a charge-coupled device IVIS imaging camera (Xenogen, Alameda, Calif.). Photons were collected for a period of 1-60 s, and images were obtained by using LIVING IMAGE 2.60.1 software (Xenogen). Tumor signal (i.e. total number of photons collected) was quantified using IGOR Pro 4.09A image analysis software (WaveMatrics, Lake Oswego, Oreg.). The tumor signal from the right mammary gland was divided by the tumor signal from the left mammary gland, and the tumor signal ratio at one week after cell transplantation (day 0) was arbitrarily set to one. The ratios of tumor signal at other time points were derived by dividing the calculated tumor signal with the tumor signal at week one, and the results were presented as mean±standard error of the mean (SEM). Forty days after initiating bioluminescence imaging, mice were sacrificed and tumors were removed and weighed. Next, the tumors were examined by western blot analysis for EGLN2 and cyclin D1 protein levels as described above in Example 1.

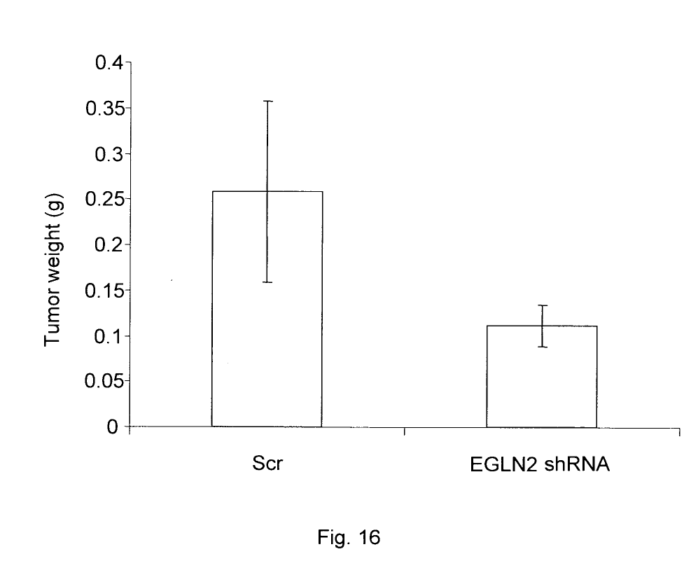

FIGS. 14A and 14B show bioluminescent images taken from a representative mouse over 40 days. FIG. 15 shows the average ratio of tumor signal of the EGLN2 shRNA tumor to the tumor signal of the control shRNA tumor. As shown in FIGS. 14A, 14B, and 15, over time, there was a progressive decline in the EGLN2 shRNA tumor signal relative to the control shRNA tumor signal, demonstrating a continued expansion of the tumors formed by the control shRNA cells and an apparent arrest of tumor growth of the EGLN2 shRNA cells. As shown in FIG. 16, tumor weight of tumors formed by the EGLN2 shRNA cells (EGLN2 shRNA) was decreased compared to tumor weight observed in tumors formed by the control shRNA (Scr).

These results showed that shRNA directed against EGLN2 decreased tumor formation and tumor weight following orthotopic implantation of human breast cancer cells in mice. These results indicated that methods and agents of the present invention are useful for reducing or decreasing tumor formation or tumor weight in a subject. These results further suggested that methods and agents of the present invention are useful for treating or preventing cancer associated with elevated levels or activity of cyclin D1 in a subject.

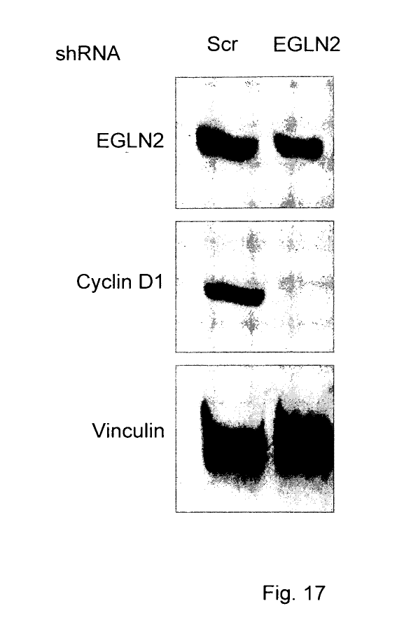

As shown in FIG. 17, breast tumors infected with shRNA directed against EGLN2 had reduced EGLN2 protein levels compared to EGLN2 protein levels observed in breast tumors infected with control shRNA (Scr). These results indicated that the shRNAs directed against EGLN2 mRNA were effective at reducing EGLN2 expression. Breast tumors infected with shRNA directed against EGLN2 had reduced cyclin D1 levels compared to those observed in breast tumors infected with control shRNA (Scr).

These results showed that shRNA directed against EGLN2 mRNA reduced cyclin D1 protein levels in breast tumors. Taken together, these results showed that methods and agents of the present invention are useful for reducing or decreasing tumor formation or tumor weight in a subject by reducing cyclin D1 levels or activity. These results further suggested that methods and agents of the present invention are useful for treating or preventing cancer associated with elevated levels or activity of cyclin D1 in a subject.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method for decreasing the level of cyclin D1 in a subject in need thereof, the method comprising administering to the subject an effective amount of an agent that inhibits the activity or expression of a prolyl hydroxylase, thereby decreasing the level of cyclin D1 in the subject.

2. The method of claim 1, wherein the prolyl hydroxylase is EGLN2.

3. A method for treating a disorder associated with elevated cyclin D1 levels or expression in a subject, the method comprising administering to the subject an effective amount of an agent that inhibits the activity or expression of a prolyl hydroxylase.

4. The method of claim 3, wherein the prolyl hydroxylase is EGLN2.

5. The method of claim 3, wherein the disorder is a cancer.

6. The method of claim 5, wherein the cancer is selected from the group consisting of an estrogen-receptor positive cancer, an estrogen-dependent cancer, and a cancer resistant to endocrine therapy.

7. The method of claim 1 or claim 3, wherein the agent is a siRNA or a shRNA comprising the nucleotide sequence of SEQ ID NO. 1 or SEQ ID NO:2.

8. A method for identifying a modulator of cyclin D1 levels, the method comprising:
(a) measuring the activity of a prolyl hydroxylase in the presence and in the absence of a candidate modulator under conditions suitable for the prolyl hydroxylase to hydroxylate a polypeptide substrate in the absence of the candidate modulator;
(b) comparing the activity of a prolyl hydroxylase measured in the presence and in the absence of a candidate modulator in step (a); and
(c) identifying the candidate modulator as a modulator of cyclin D1 levels if the activity of the prolyl hydroxylase differs in the presence and in the absence of the candidate modulator.

9. The method of claim 8, wherein the prolyl hydroxylase is EGLN2.

10. The method of claim 8, wherein the activity of the prolyl hydroxylase is determined by measuring prolyl hydroxylation of the polypeptide substrate.

11. The method of claim 10, wherein the polypeptide substrate is the alpha subunit of hypoxia-inducible factor or a fragment thereof containing a proline residue.

12. The method of claim 10, wherein measuring the hydroxylation of the polypeptide substrate comprises measuring the binding of a VHL polypeptide to the polypeptide substrate.

13. A method for identifying a modulator of cyclin D1 levels, the method comprising:
(a) measuring the activity of a prolyl hydroxylase in the presence and in the absence of a candidate modulator under conditions suitable for the prolyl hydroxylase to hydroxylate a polypeptide substrate in the absence of the candidate modulator;
(b) comparing the activity of a prolyl hydroxylase measured in the presence and in the absence of a candidate modulator in step (a);
(c) identifying the candidate modulator as one that alters the activity of the prolyl hydroxylase if the activity of the prolyl hydroxylase differs in the presence and in the absence of the candidate modulator;
(d) measuring the levels of cyclin D1 in the presence and in the absence of the candidate modulator identified in step (c);
(e) comparing the levels of cyclin D1 measured in the presence and in the absence of the candidate modulator in step (d); and
(f) identifying the candidate modulator as a modulator of cyclin D1 levels if the level of cyclin D1 differs in the presence and in the absence of the candidate modulator.

14. The method of claim 13, wherein the prolyl hydroxylase is EGLN2.

15. The method of claim 13, wherein the activity of the prolyl hydroxylase is determined by measuring prolyl hydroxylation of the polypeptide substrate.

16. The method of claim 15, wherein the polypeptide substrate is the alpha subunit of hypoxia-inducible factor or a fragment thereof containing a proline residue.

17. The method of claim 15, wherein measuring the hydroxylation of the polypeptide substrate comprises measuring the binding of a VHL polypeptide to the polypeptide substrate.

18. A method for identifying a modulator of EGLN2 expression or activity, the method comprising:
(a) measuring the levels of cyclin D1 in the presence and in the absence of a candidate modulator;
(b) comparing the levels of cyclin D1 measured in the presence and in the absence of a candidate modulator in step (a); and
(c) identifying the candidate modulator as a modulator of EGLN2 expression or activity if the levels of cyclin D1 differs in the presence and in the absence of the candidate modulator.
